(19) United States
(12) Patent Application Publication
(10) Pub. No.: US 2010/0135937 A1
(43) Pub. Date: Jun. 3, 2010

(54) METAL OXIDE NANOCRYSTALS: PREPARATION AND USES

(75) Inventors: Stephen O'Brien, New York, NY (US); Limin Huang, Jersey City, NJ (US); Zhuoying Chen, Cambridge (GB); Ioannis Kymissis, New York, NY (US); Zhang Jia, New York, NY (US)

Correspondence Address: SCHWEGMAN, LUNDBERG & WOESSNER/COLUMBIA UNIVERSITY, P.O. BOX 2938, MINNEAPOLIS, MN, 55402, US

(73) Assignee: The Trustees of Columbia University in the City of New York, New York, NY (US)

(21) Appl. No.: 12/566,135

(22) Filed: Sep. 24, 2009

Related U.S. Application Data
(63) Continuation-in-part of application No. PCT/US2008/003878, filed on Mar. 25, 2008.
(60) Provisional application No. 60/920,004, filed on Mar. 26, 2007.
(60) Provisional application No. 60/908,081, filed on Mar. 26, 2007.
(60) Provisional application No. 61/210,481, filed on Mar. 19, 2009.

(51) Int. Cl. A61K 8/19; H01L 51/30; H01L 27/07; H01L 29/92; H01G 9/00; H01G 4/06; C09K 3/00; C01B 13/00; C01G 25/02; C01F 17/00; C01G 45/12; C01G 49/02; C01G 53/04; C01G 51/04; C01G 23/04; C01G 19/02; C01G 41/02; C01G 11/02; C01G 31/02; B32B 27/32; H01L 21/302; B05D 5/12

(52) U.S. Cl. 424/59; 257/40; 257/532; 257/300; 361/502; 361/524; 361/311; 252/182.32; 252/182.33; 423/593.1; 423/594.12; 423/263; 423/599; 423/594.1; 423/594.3; 423/594.5; 423/598; 423/594.9; 423/594.13; 423/594.14; 423/594.8; 428/220; 438/758; 427/126.3; 257/E51.049; 257/E27.034; 257/E29.342; 977/773; 977/762; 257/E21.214

(57) ABSTRACT

Nanocrystalline forms of metal oxides, including binary metal oxide, perovskite type metal oxides, and complex metal oxides, including doped metal oxides, are provided. Methods of preparation of the nanocrystals are also provided. The nanocrystals, including uncapped and uncoated metal oxide nanocrystals, can be dispersed in a liquid to provide dispersions that are stable and do not precipitate over a period of time ranging from hours to months. Methods of preparation of the dispersions, and methods of use of the dispersions in forming films, are likewise provided. The films can include an organic, inorganic, or mixed organic/inorganic matrix. The films can be substantially free of all organic materials. The films can be used as coatings, or can be used as dielectric layers in a variety of electronics applications, for example as a dielectric material for an ultracapacitor, which can include a mesoporous material. Or the films can be used as a high-K dielectric in organic field-effect transistors. In various embodiments, a layered gate dielectric can include spin-cast (e.g., 8 nm-diameter) high-K BaTiO₃ nanocrystals and parylene-C for pentacene OFETs.

### CROSS-REFERENCE TO RELATED APPLICATIONS

This application is a continuation-in-part of PCT/US2008/003878, published as WO2008/118422, the priority of which is claimed and the disclosure of which is incorporated by reference herein in its entirety, which claims the priority of U.S. Ser. No. 60/920,004, filed Mar. 26, 2007, and of U.S. Ser. No. 60/908,081, filed Mar. 26, 2007, which are incorporated herein by reference in their entireties. This application also claims the priority of U.S. Ser. No. 61/210,481, filed Mar. 19, 2009, which is incorporated by reference herein in its entirety.

### STATEMENT OF GRANT SUPPORT

This invention was made with government support under award numbers DMR-0213574, CHE-011752, CHE-0641523, and ECCS-0644656 from the National Science Foundation (NSF) and award number DE-FG02-03ER15463 from the Department of Energy (DoE), and by the New York State Office of Science, Technology, and Academic Research (NYSTAR), and relied on equipment supported by the NSEC program of the National Science Foundation under Award Number CHE-0117752, and by a CAREER award, DMR-0348938. The government has certain rights in this invention.

### BACKGROUND

Nanoparticles including metal oxides are of considerable interest. Accordingly, methods of preparation have been studied. For example, U.S. Pat. No. 6,262,129 describes methods of preparation of metal nanoparticles (e.g., cobalt) using surfactants, wherein the metal nanoparticles have comparatively narrow size distributions.

Among nanoparticles composed of metal oxides, certain materials are of interest. Complex oxide perovskites have been of interest for more than half a century due to their ferroelectric, pyroelectric, piezoelectric and dielectric properties¹⁻². Their applications in the electronics industry include transducers and actuators,³ high-K dielectric capacitors,⁴ and memory applications, such as in ferroelectric random access memories (FRAMs), which rely on the existence of a spontaneous polarization in the crystal unit cell.⁵⁻⁷ Since the physical properties of materials in the nanoscale regime (1-100 nm) can be quite different from the bulk⁸⁻¹°, and the precise nature of ferroelectricity at the nanoscale is still debated,¹¹ interest has been stimulated over the preparation and study of complex oxide perovskite nanocrystals. Of particular interest is the nature of the phase transition temperature (T_{C}, called the Curie temperature) that marks the transition between the ferroelectric and paraelectric phase, respectively.¹²⁻¹³ This transition is known to be size dependent for the ferroelectric perovskites at the nanoscale.¹²⁻¹³ Uniform, monodisperse and highly crystalline nanoparticles with tunable sizes and morphologies are desired in order for a consensus to be reached on the exact nature of critical size suppression of ferroelectricity.

As a prototype and model system of ferroelectric perovskite crystals, barium titanate nanostructures, including thin films, nanocrystals, nanowires and nanotubes, have been synthesized by a wide variety of approaches in the literature. Synthesis methods mainly include non-chemical and chemical methods. Non-chemical methods, including Pulsed Laser Deposition (PLD)^{14-20,26-27} and magnetron sputtering, ²¹⁻²⁵ have successfully deposited well crystalline epitaxial BaTiO₃ thin films on different substrates. Some of these epitaxial films provide well-behaved electrical and optical properties and they have been employed for the fabrication of dynamic random access memories (DRAM), electro-optical devices and thin film capacitors.^{14,19,26-27} Chemical approaches have also been widely studied in the synthesis of BaTiO₃ due to the desire to understand fundamentally the relationship between the particle size and ferroelectricity and to reduce the cost to produce ferroelectric nanostructures. Chemical approaches also offer an advantage for potential nanocrystal self-assembly. Chemical approaches for the synthesis of BaTiO₃ nanostructures include sol-gel processing,²⁸⁻³⁵ coprecipitation,^{36-39,55} pyrolysis⁴⁻⁴² and hydrolysis⁴³⁻⁴⁴ of metallo-organic or bimetallic alkoxide precursors, hydrothermal⁴⁵⁻⁵¹ or solvothermal⁵²⁻⁵³ synthesis and peptide templates assisted room temperature synthesis.⁵⁴ Most can be classified as aqueous synthesis and only a few of them⁵²⁻⁵⁴ are considered nonaqueous (non-hydrolytic). In nonaqueous synthesis, it is generally easier to control the nanocrystal size distribution for uniform nanocrystals. In certain cases the nanocrystals are prepared with surface capping ligands.^{43-44,50,55} Without surface capping ligands aggregation is a major problem which creates difficulties for physical property measurements, although nanocrystals can be temporally dispersed into solvent by strong sonication. With surface bound ligands, nanocrystals can be well dispersed into solvents and functionalized by conjugation of functional groups to surface ligands or by ligand exchange. Furthermore, uniform and well-dispersed nanocrystals could be used in self-assembly, to create nanocrystal superlattices that have potentially interesting collective opto-electronic properties.⁵⁶⁻⁵⁷

Mesoporous structures (mesostructured materials) include porous inorganic and inorganic/organic hybrid ultra high surface materials for catalysis, surface functionalization, and electronic/optoelectronic use. The technological backbone is a process of forming high surface area mesostructured materials (materials containing pores with diameters between 2 and 50 nm). See for example U.S. Pat. No. 7,176,245 by SBA Materials Inc.

Capacitors are the devices which can store charge (hence energy) in a small area. Typically capacitors are either electrochemical type (where electrolyte ions store energy) or solid state type (where electrons store energy). Energy storage ability of a capacitor depends upon it's capacitance per unit area and the voltage it can sustain. Capacitance depends upon the dielectric constant of the material used (as insulating layer) and surface area of material where charge is stored.

Higher dielectric materials are preferable so that capacitor can sustain higher voltages before breakdown. Traditional capacitor research is mostly focused on improving the dielectric material. Recent advances in high surface area nanomaterials have resulted in new materials which can store more charge in a highly porous 3-d configuration as oppose to a simple metal plate, this has lead to the development of ultracapacitors (or supercapacitors)

Ultracapacitors are based on a structure that contains an electrical double layer. In a double layer, the effective thickness of the “dielectric” is exceedingly thin—on the order of nanometers and that, combined with the very large surface area, is responsible for their extraordinarily high capacitances in practical sizes. Ultracapacitors can have power densities (energy density stored/delivered per unit time) which are 10 to 100 times higher than conventional batteries. They can also have a very nigh number of charge-discharge cycles, millions or more compared to 200-1000 recharges for most commercially available rechargeable batteries. The efficiency of ultracapacitors compared to batteries is also high. Ultracapacitors offer promise for hybrid automotive engines, starter batteries, consumer electronics, and UPS power supplies.

Organic field-effect transistors (OFETs) are promising components for large-area electronics. OFETS can offer simple fabrication, potential for low cost fabrication, large-area processability, and, by some measures, superior device performance to amorphous silicon. Improved device performance can be desirable.

Effort can be applied to improving device performance, such as primarily by focusing on enhancing device mobility or improving sub-threshold behavior. One approach to improving both such figures of merit can be to use a high-capacitance gate dielectric. This can reduce the operating voltage and can increase the mobile charge carrier density for a given gate voltage. Operating at a higher channel charge density can improve the effective mobility in an OFET, such as by filling deeper trap states and allowing carrier conduction in states that are further from the mobility edge. Several approaches for increasing the gate dielectric capacitance can be proposed, such as including the use of very thin dielectrics, deposition of a high-K inorganic dielectric such as via a sputtering or sol-gel process followed by high-temperature annealing, and the use of a high-K (e.g., <15) polymer dielectric. These approaches can introduce several process complications, for example: thin layers can require highly demanding surface conditions, use of inorganic gate dielectrics from solution can require a high-temperature, sputtering can require potentially high-cost vacuum processing, and a polymeric gate material can offer a limited dielectric constant. In an example of an approach, the dispersion of high-K (e.g., >30) nanocrystals in a polymer can allow solution processing, which can provide the potential for a high dielectric constant, but can also suffer from polarization hysteresis at the nanoparticle/polymer interface and a limited nanocrystal loading fraction leading to a limited dielectric constant.

### SUMMARY

Metal oxide nanocrystalline forms, methods of preparation of the nanocrystalline forms, stable dispersions of the nanocrystalline forms and methods of preparation thereof, uses for the nanocrystalline forms and dispersions thereof, including films incorporating the nanocrystalline forms, methods of formation of films including the use of dispersions of the nanocrystalline forms, and uses for the films, are disclosed and claimed herein.

In various embodiments of the disclosed subject matter, a nanocrystalline form of a metal oxide, the form comprising a plurality of nanocrystals, the plurality of nanocrystals having a narrow size distribution and an average particle diameter ranging from about 1 nm to about 100 nm, the nanocrystals comprising a metal oxide of formula M¹ₓO_{z}, a mixed metal oxide of the perovskite type of formula M²M³O₃, or a complex mixed metal oxide of the formula M⁴ₓM⁵_{y}O_{z}, wherein all of M¹-M⁵ are independently selected ions of metallic elements, are provided. In various embodiments, the nanocrystals can be uncapped and uncoated, or can be capped or coated with an organic coating material. In various embodiments, the narrow size distribution is a monodisperse size distribution, for example having a monodispersity of <10%.

In various embodiments, a method of preparation of the metal oxide nanocrystalline form, comprising contacting an metalorganic precursor, wherein the metalorganic precursor comprises a single metallic element or more than one metallic element, and a liquid substance comprising an alcohol at an elevated temperature of less than about 350° C., to provide the plurality of metal oxide nanocrystals having a narrow size distribution, are provided. Optionally, a reagent can be put in contact with the metalorganic precursor and liquid substance, following application of the elevated temperature, to provide a plurality of metal oxide nanocrystals. The metal oxide precursor solution can be formed using a metalorganic compound including a single metallic element or including a plurality of metallic elements.

In various embodiments, a substantially homogeneous dispersion of the nanocrystalline form, or a substantially homogeneous dispersion of a nanocrystalline form prepared by an inventive method, in a liquid, is provided.

In various embodiments, methods of preparation of substantially homogeneous dispersions are provided. The dispersions can be stable over a period of time.

In various embodiments films including the inventive nanocrystals, or including nanocrystals prepared by an inventive method, or using an inventive dispersion or a dispersion prepared by an inventive method, are provided. The films can include various matrix materials, such as organic, inorganic, or mixed organic/inorganic matrix materials, in combination with the metal oxide nanocrystals. The films can have high dielectric constants. The films can have various physical properties that are substantially unchanged from the properties of the respective types of nanocrystals from which the films are formed.

In various embodiments, inventive nanocrystals or thin films incorporating inventive nanocrystals can be incorporated into various devices such as capacitors, ultracapacitors, semiconductor devices, optoelectronic devices, and display devices.

In various embodiments, organic field effect transistors comprising nanoparticles or films as disclosed and claimed herein are provided.

### BRIEF DESCRIPTION OF THE FIGURES

FIG. 1 shows TEM and XRD analysis of as synthesized 6-10 nm BaTiO₃ nanocrystals capped with decanoic acid (case I). a) Overview; b) selected area electron diffraction (SAED); c) XRD powder patterns. All reflections can be assigned to the BaTiO₃ phase (JCPDS No. 31-174); d) high resolution TEM (HRTEM) image of an individual nanocrystal on the <111> zone axis.; e) HRTEM image of an individual nanocrystal on the <100> zone axis; f) power spectrum (PS) of (d); g) PS of (e).

FIG. 2 shows TEM and XRD analysis of as synthesized 3-5 nm BaTiO₃ nanocrystals capped with oleic acid (case II). a) Overview; b) selected area electron diffraction (SAED); c) XRD powder patterns. All reflections can be assigned to the BaTiO₃ phase (JCPDS No. 31-174); d) high resolution TEM (HRTEM) image of an individual nanocrystal on the <111> zone axis.; e) HRTEM image of an individual nanocrystal on the <110> zone axis; f) power spectrum (PS) of (e).

FIG. 3 shows TEM and XRD analysis of as synthesized 10-20 nm BaTiO₃ nanoparticle and nanorod mixture capped with oleyl alcohol (case III). a) Overview; b) a typical nanoparticle; c) a typical nanorod; d) selected area electron diffraction (SAED); e) XRD powder patterns. All reflections can be assigned to the BaTiO₃ phase (JCPDS No. 31-174).

FIG. 4 shows IR studies of four samples: (a) benzyl alcohol (99.8% Aldrich); (b) Ba dissolved in benzyl alcohol (Ba benzyl alcoholate in benzyl alcohol); (c) titanium (IV) isopropoxide Ti[OCH(CH₃)₂]₄ (99.999% Aldrich); and (d) the as synthesized precursor in case I.

FIG. 5 shows a ¹H-NMR spectrum of a filtered bimetallic (Ba, Ti) metalorganic precursor solution.

FIG. 6 shows XRD patterns of BaTiO₃ nanocrystals synthesized in a solvothermal process using metal barium and titanium isopropoxide as a precursor. The solvothermal process were carried out under different alcohols and alcohol mixture (a) ethanol; (b) 95% ethanol; (c) ethanol+isopropanol (volume ratio of 1:1); and (d) isopropanol.

FIG. 7 shows XRD patterns of BaTiO₃ nanocrystals synthesized in a solvothermal process using BaTi ethylhexano-isoproxide as a precursor. The solvothermal process were carried out under different alcohols (a) ethanol and KOH; (b) 95% ethanol; (c) isopropanol; and (d) ethanol.

FIG. 8 shows TEM images of BaTiO₃ nanocrystals synthesized in a solvothermal process using different alcohols and BaTi metalorganic sources. (a) ethanol; (b) isopropanol; (c) 95% ethanol; (d) ethanol+isopropanol. (a-d) metal barium and titanium isopropoxide as a precursor. (e) isopropanol; and (f) 95% ethanol. (e,f) BaTi ethylhexano-isoproxide as a precursor.

FIG. 9 shows photographic images of oleic acid coated BaTiO₃ nanocrystals with different sizes dispersed in hexane to afford homogeneous, and transparent/semitransparent nanocrystal suspension.

FIG. 10 shows Scanning Electron Microscopy (SEM) images of BaTiO₃ (BT) thin films composed of the nanocrystals with two different sizes, respectively (top-view). The films were prepared by spin-coating of a hexane suspension of the oleic acid coated nanocrystals. Various film thickness can be achieved applying multiple spin-coating (inset, cross-sectional view).

FIG. 11 shows: (a) Photo image of a stable ethanol solution of BaTiO₃ nanocrystals; (b) TEM image of corresponding individual BaTiO₃ nanocrystals; (c,d) BaTiO₃ nanocrystal thin film by multiple spin-coating (five coatings), top view and cross-sectional view, respectively.

FIG. 12 shows the thermal stability of BaTiO₃ nanocrystal thin films at different temperatures, as discussed below. There is no diffraction peak sharpening for samples annealed at temperatures lower than 600° C. Significant peak sharpening can be observed at a higher temperature of 800° C., indicating the crystal growth.

FIG. 13 shows polarization-electric field curve based on the C−V measurement of the BaTiO₃ nanocrystal (˜8 nm in diameter) thin film (five coatings).

FIG. 14 shows several images: (a) SEM image of a micro-patterned BaTiO₃ nanocrystal thin films prepared by MIMIC with a micro-patterned PDMS stamp; (b) High-resolution SEM image of BaTiO₃ nanocrystal thin film prepared by spin-coating, showing that the thin film is composed of nanocrystals with a uniform size of ˜10 nm; (c) Tapping mode AFM image of the BaTiO₃ nanocrystal thin film. All these films were prepared on n-Si substrates with native oxide layers.

FIG. 15 shows frequency dependence (1 KHz to 100 KHz) of the dielectric constant and dielectric loss of the nanocrystal BaTiO₃ 108 nm thin film at room temperature.

FIG. 16 illustrates an example of frequency dependent dielectric constant for BaTiO₃/parylene. The inset in FIG. 1 is a TEM image of 8 nm BaTiO₃ nanocrystals (the scale bar represents 100 nm).

FIG. 17 illustrates a SEM photograph of pentacene grown on (a) bare BaTiO₃ thin film and (b) parylene C coated BaTiO₃ thin film. The scale bars in both images represent 1 μm.

FIG. 18 is an example of a plot of drain-source current I_{DS} (represented by solid squares), \sqrt{I_{Ds}} (represented by open squares) and gate leakage current I_{GS} (represented by open circles) versus gate-source voltage V_{GS} of the BaTiO₃/parylene OFETs in the saturation region.

FIG. 19 is an example of a graph of mobility vs. gate-source voltage, illustrating linear mobility in OFETs with 110 nm parylene only (represented by dots) as gate dielectric and the ones with BaTiO₃/parylene (represented by squares) as gate dielectric. The capacitance of 100 nm parylene-C is 25 nF/cm², and that of the composite dielectric is 31 nF/cm². The inset in FIG. 4 illustrates QSCV of BaTiO₃/parylene OFET.

### DETAILED DESCRIPTION

A nanoparticle is a physical form of a solid material wherein the individual particle dimensions are of the order of nanometers (10⁻⁹ meters), ranging up to no more than about 1 micron (10⁻⁶ meters) in average diameter, typically less than 100 nm (10⁻⁷ meters). As the term is used herein, a “nanocrystal” is a nanoparticle composed of a single crystal domain or a nanocomposite of multiple crystal domains within an individual nanoparticle. The nanocrystals disclosed and claimed herein are composed of a metal oxide or a mixed metal oxide, or combinations thereof, as defined. For example, a nanocrystal herein can be composed of a member of the perovskite family of minerals or of other members of this class of metal oxides of the general formula M²M³O₃ wherein M² and M³ are metal ions. Examples include barium titanate (BaTiO₃) and lead titanate (PbTiO₃). A nanocrystal can be composed of a binary metal oxide of the general formula M¹ₓO_{z} wherein M¹ is a metal ion. Examples include zinc oxide, zirconium oxide, and titanium oxide. Alternatively, a nanocrystal can be composed of a complex metal oxide of the general formula M⁴M⁵_{y}O_{z}, wherein M⁴ and M⁵ are metal ions. Examples include indium tin oxide and lithium niobium oxide. A nanocrystal can also include ions of additional metallic elements other than the predominant metal ions selected from M¹-M⁵ as defined above in a crystal lattice of the metal oxide, mixed metal oxide, or complex mixed metal oxide. Examples include ions of zirconium, yttrium, or rare earth metals. Accordingly, a nanocrystal can include both the metallic elements making up the bulk oxide, and can also include dopants of various ions of other metallic elements, or can include metal oxide forms where more than a single type of metallic element can occupy a particular crystal site. For example, nanocrystals of (cobalt, manganese)-doped zinc oxide include the binary oxide zinc oxide, but additionally containing ions of cobalt and manganese in various proportions. All such compositions of this type are included in the disclosed subject matter.

A nanocrystal can have an average diameter of about 1 to about 100 nm. A collection or plurality of a particular type of nanocrystal composed of a type of material, referred to herein as a “nanocrystalline form” of that type of material, for example a material prepared by a disclosed method, can have a narrower size distribution within this range of sizes. For example, in a particular sample, the nanocrystals can be predominantly of a size of about 2-3 nanometers, or about 10-20 nanometers. Within a particular sample, the range of individual particle sizes can be monodisperse, indicating a normal distribution of particle sizes around a mean. A nanocrystalline form having a narrow size distribution, a monodisperse size distribution, or both, can provide uniform sample properties. Methods as disclosed and claimed herein provide nanocrystalline forms wherein the size distribution is narrow, and can be monodisperse, for example with a monodispersity of <10%. The average nanocrystal particle size in a particular sample is tunable using the methods herein, such that nanocrystalline forms of various compositions, average particle diameters, and particle size distributions can be prepared by these methods.

The solid nanoparticles, nanocrystals herein, need not be of any particular shape. Nanocrystals can be roughly spherical, or can be elongate, or can form regular or irregular polyhedra. An “average particle diameter” as the term is used herein refers to a numerical average of x, y, and z orthogonal axes that, when not all equal as in a roughly spherical nanocrystal, are defined with x being the longest dimension of the particle.

A nanocrystal can be “uncoated” and “uncapped”, that is, not having a distinct surface layer composed of a different material, such as an organic material. Alternatively, a nanocrystal can be “coated” or “capped”, meaning that the layer of the material on the surface of each nanocrystal can include other materials in addition to the bulk material of the crystalline phase. The term “capped nanocrystal” refers to an inventive nanocrystal wherein the nanocrystal is covered with a molecular layer of one or more organic compounds, that it bonded to the surface of the metal oxide nanocrystal, typically by non-covalent interactions. A “coated” nanocrystal includes a capped nanocrystal as well as including an inventive metal oxide nanocrystal where more than a single molecular layer covers the nanocrystal surface; a coated nanocrystal can have many molecular layers of an organic compound or a mixture of organic compounds on its surface. The terms “capped” and “coated” refer to nanocrystals having on the surface an “organic coating material” that is not volatile to any great extent and which requires heating to a relatively high temperature, such as in sintering, to remove the organic material. The nanocrystals can be capped with a “ligand,” which refers to an organic molecule capable of complexing, or forming typically non-covalent bonds with, an inorganic molecular entity. For example, a nanocrystal, for example barium titanate, can be capped with a ligand such as oleic acid, a hydrophobic long chain with a carboxyl end group. An “uncapped” or “uncoated” nanocrystal can have a dry surface, or can have a surface wetted with a volatile liquid such as an alcohol or water, wherein the volatile liquid can be removed if necessary without resorting to elevated temperatures, for example temperatures over 100° C. An “uncapped” or “uncoated” nanocrystal also includes a nanocrystal wherein a surface layer of the metal oxide also includes metal alkoxide or metal hydroxide groups, or a layer of a volatile solvent such as an alcohol or ether.

A “metallic element” as the term is used herein refers to the identity of the element in any chemical form, i.e., a metallic element is an element classified as metallic (i.e., not a non-metal such as oxygen, etc.) which can be in the form of an elemental metal (zero-valent) or can be in form of a salt or complex of a metal (metal ion). A metallic element in metallic form refers to the unoxidized metal, whereas a metallic element in salt or complex form refers to the metal in chemical combination with other elements, such as forming ionic bonds with other elements. For example, the compound barium titanate comprises two metallic elements barium and titanium, each element being in a salt or complex form, namely in an oxide form wherein each metal is in an elevated oxidation state, i.e., an oxidation state of +1 or higher.

A “metal oxide”, “mixed (or doped) metal oxide”, or “complex mixed metal oxide” can each include various species wherein the metal ion, although comprising an identical metallic element, is in a different oxidation state in distinct species, and thus is composed with a different stoichiometry. For example, a “manganese oxide” herein can include manganese(II) oxide (MnO), manganese(III) oxide (Mn₂O₃), manganese dioxide (manganese(IV) oxide, MnO₂), manganese trioxide (manganese(VI) oxide, MnO₃), manganese(VII) oxide (Mn₂O₇), any other stable manganese oxide, or a combination; an “iron oxide” can include FeO and Fe₃O₄, or any other stable oxides, or a combination.

A “rare earth” metallic element comprises the elements of the lanthanides, elements 57-71, as is well known in the art.

A “metalorganic precursor” or a solution thereof refers to a molecular composition including at least one metallic element that can be reacted with a liquid comprising an alcohol, such as 95% ethanol, or isopropanol, or an isopropanol-water mixture, or an alcohol-water mixture containing an alkali, to provide nanocrystals. Examples of metalorganic precursors include metal alkoxides, metal carboxylates such as metal acetates, and metal complexes such as metal acetoacetonates. Metalorganic precursors can be present as solutions in solvents of various types to provide a metalorganic precursor solution. A metalorganic precursor solution can be contacted with an alcohol or a liquid comprising an alcohol and optionally, water and/or an alkali, at an elevated temperature of less than about 350° C., to provide a nanocrystalline form. This process can be termed a “solvothermal” process, as it involves solvents and heat. The nanocrystalline form can be recovered in solid form by contact with a reagent. Alternatively, the nanocrystalline form can be handled as a stable dispersion in a liquid medium. The nanocrystalline form can be uncapped and uncoated, i.e., lacking organic surface layers. Optionally, metalorganic precursor solution can include an organic material that provides a capping or coating material. Examples are fatty acids, fatty amines, and fatty alcohols.

A “reagent” as the term is used herein refers to a liquid material that tends to induce formation of nanocrystals or separation of nanocrystals from a suspension or dispersion in another liquid material. For example, nanocrystals can be formed by contacting a metalorganic precursor and a liquid comprising an alcohol at an elevated temperature. Following application of the elevated temperature to the reaction mixture, nanocrystals are present in the liquid milieu. The nanocrystals can be collected, such as by centrifugation. However, addition of a reagent can assist in techniques for recovering the nanocrystals from the reaction mixture, bringing about additional nanocrystal formation or increasing the ease of separation of the nanocrystals, or both.

A “dispersion” as the term is used herein refers to a mixture of the plurality of nanocrystals and a liquid wherein the predominant portion of the nanocrystals are suspended in the liquid such that they do not readily precipitate out. A dispersion within the meaning herein is stable for a period of time, which can range from about one hour to about six months. Another term for a dispersion is a “suspension”. A certain amount of precipitation can occur, and the dispersed solid nanocrystals can be caused to precipitate by centrifugation, but under normal gravitational conditions they do not rapidly collect at the bottom of a vessel containing the dispersion. A dispersion can be transparent, or can be opalescent, or cloudy, depending at least in part on the size of the dispersed nanocrystals. The liquid in which the plurality of nanocrystals is dispersed can be an organic solvent, such as a non-polar organic solvent. Examples are hydrocarbons (aliphatic or aromatic), chlorocarbons, and the like. The organic solvent can also be a polar solvent, such as acetone, an alcohol, an ether, or a mixture of a water-miscible solvent and water. The liquid can also be a mixture of various types of organic solvents, and optionally water. Alternatively the liquid can be supercritical carbon dioxide.

The term “not prone to aggregation or clumping” means herein that the individual nanocrystals do not attract each other such that, for instance, a dispersion of the nanocrystals in a liquid substance is stable over a period of time, the nanocrystals do not rapidly form larger aggregates, and the nanocrystals remain in suspension. It is well known in the art that nanoparticles such as nanocrystals, particularly those that lack capping or coating groups, tend to attach each other and form larger clumps of the material. However, the disclosed nanocrystals, including those that are uncapped and uncoated, have a greater propensity to remain dispersed and distinct than do art nanoparticles.

A “copolymer” refers to a polymeric material, as is well known in the art, that includes two or more types of monomers. A “block copolymer” is a copolymer wherein the two or more types of monomers are incorporated within distinct oligomeric moieties, which are bonded to each other to create a copolymer with blocks of each type of monomeric unit.

The term “P₁₂₃” as used herein refers to a triblock copolymer surfactant in the Pluronic® group.

A “mesoporous material” refers to a type of porous materials containing ordered pore structure with diameters between 2 and 50 nm, according to IUPAC notation. The matrix is typically amorphous silica oxide (silica) based, but can be composed of other metal oxides. A silica mesoporous material can be prepared by hydrolysis and condensation of tetraethyl orthosilicate templated by supermolecular arrays of surfactant (micellar rods). After the organic-inorganic composites are formed, the organic templates can be removed by thermal treatment or solvent extracting, leaving behind an inorganic matrix with ordered pores and channels corresponding to the original surfactant supermolecular templates. Based on the type and size of surfactant templates being used, the pore sizes can vary and the channel structure can be one-dimensional or three-dimensional. There are two main series of mesoporous materials, MCM (Mobil Company Materials: MCM-41, one-dimensional; MCM-48, three-dimensional), and SBA series (Santa Barbara Amorphous: SBA-15, one-dimensional; SBA-16, three-dimensional). Mesoporous materials have order pore structure, high surface area and high porosity, and can be applied to fields such as catalysis, sorption, and electronics. See, for example, U.S. Pat. No. 7,176,245 and documents cited therein.

By “a type of the nanocrystalline form” as the phrase is used herein is meant a particular elemental composition, average particle diameter, particle diameter distribution, and other characteristic attributes of an inventive nanocrystalline form. When it is stated, for example, that a film comprising a nanocrystalline form “substantially retains” some property “of the respective type of nanocrystalline form”, what is meant is that the particular property in question, such as dielectric constant, density, spectral absorption or reflectivity, etc., is not greatly altered, diminished, etc. through the process of incorporation of the nanocrystalline form into the corresponding film.

A “matrix” as the term is used herein refers to a solid composition, formed by polymerization or condensation of a “matrix precursor”, which can be a solid or a liquid, that incorporates nanocrystals and is cohesive, holding the nanocrystals embedded in the matrix, which can serve to provide a cohesive film. A “film” as the term is used herein refers to a physical structure wherein the thickness is substantially less than the length or breadth; a film can be flat or can be curved in various forms. A film can coat an underlying structure or surface, such as glass, metal, human skin, plastic, and other types of surfaces. A film can be disposed on a substrate such as a sheet or wafer composed at least in part of silicon, silica, silicon nitride, or diamond.

The disclosed subject matter provides a nanocrystalline form of a metal oxide, the form comprising a plurality of nanocrystals, the plurality of nanocrystals having a narrow size distribution and an average particle diameter ranging from about 1 nm to about 100 nm, the nanocrystals comprising a metal oxide of formula M¹ₓO_{z}, a mixed metal oxide of the perovskite type of formula M²M³O₃, or a complex mixed metal oxide of the formula M⁴M⁵_{y}O_{z}, wherein all of M¹-M⁵ are independently selected ions of metallic elements.

Within any given sample disclosed herein, the average particle size can range from about 1 nm to about 100 nm, but the distribution of particle sizes around the average in a particular sample or plurality of nanocrystals is narrow. Accordingly, a particular plurality of nanocrystals can have an average particle diameter of, for example, 5 nm, and have virtually no members of that plurality having an individual particle diameter greater than, for example 8 nm, or less than about 2 nm. Thus, although samples of nanocrystals disclosed herein can have an average over an approximately 1-100 nm size range, within any particular sample, the size range is much smaller. The distribution of individual nanocrystal diameters can be substantially monodisperse, i.e., having a normal distribution around a mean, with a relatively small standard deviation compared to the mean. This deviation can be 10% or less. This implies a relatively uniform set of properties being present among the individual nanocrystals, which provides for relatively uniform bulk physical properties throughout the sample. Using the methods disclosed herein, nanocrystalline forms of a wide variety of compositions and physical parameters such as average particle diameter can be prepared, i.e., the methods are “tunable” to achieve a particular desired result.

A metal oxide of formula M¹ₓO_{z}, typically referred to as a “binary metal oxide”, wherein M₁ is a metal ion, contains predominantly one metallic element in ionic form. However, additional ions of other metallic elements can be present in the lattice of the nanocrystalline material, such as dopants. Examples of dopants include ions of ions of zirconium, yttrium, or rare earth metals. Examples of M′ in include metallic elements such as titanium, zirconium, hafnium, vanadium, niobium, tantalum, tungsten, manganese, iron, cobalt, nickel, copper, zinc, gallium, indium, tin or cerium. The particular element of a particular metal oxide nanocrystalline form can be combined with oxygen in various stoichiometries; in general, x is 1 to about 3 and z is 1 to about 6. Even for a particular metal M¹, different compositions with oxygen can exist, for example as in the cases of Mn₂O₃ and MnO₂, both of which are binary metal oxides within the meaning herein. Specific examples of binary metal oxides that can compose a nanocrystalline form of the disclosure are zinc oxide, titanium oxide, and zirconium oxide.

A metal oxide of the general formula M²M³O₃ is referred to a mixed metal oxide of the perovskite type. Again, M² and M³ are independently selected metal ions. Accordingly, perovskite type metal oxides contain predominantly two distinct metallic elements in combination with oxygen in a defined stoichiometry. Again, additional ions of other metallic elements can be present in the lattice of the nanocrystalline material, such as dopants. Examples of dopants include ions of ions of zirconium, yttrium, or rare earth metals. Examples of M² include barium, strontium, calcium, lithium, lead, yttrium, bismuth, lanthanum, or a rare earth metal. Example of M³ include titanium, zirconium, iron, copper, manganese, cerium, or cobalt. Some specific examples of perovskite type metal oxides include barium titanate, strontium titanate, calcium titanate, barium strontium titanate, barium lanthanum, lithium lanthanum titanate, lead titanate, lead zirconium titanate, barium zirconate, lead zirconate, yttrium ferrite, bismuth ferrite, yttrium barium copper oxide, lanthanum manganese oxide, strontium cerium oxide, or a rare earth cobalt oxide.

A metal oxide of the general formula M⁴, M⁵_{y}O_{z}, is referred to as a complex mixed metal oxide. Again, M⁴ and M⁵ are independently selected metal ions. The particular element of a particular metal oxide nanocrystalline form can be combined with oxygen in various stoichiometries; in general, x is 1 to about 3, y is 1 to about 5, or z is 3 to about 12, or any combination thereof. M⁴, for example, can be indium, lithium, bismuth or yttrium. M⁵, for example, can be tin, niobium, or iron. Examples of complex mixed metal oxides in nanocrystalline form of the disclosed subject matter include indium tin oxide, lithium niobium oxide, or a garnet. Examples of garnet include Bi₃Fe₅O₁₂ or Y₃Fe₅O₁₂. Again, additional ions of other metallic elements can be present in the lattice of the nanocrystalline material, such as dopants. Examples of dopants include ions of ions of zirconium, yttrium, or rare earth metals.

A nanocrystalline form of whichever metal oxide type can include nanocrystals wherein the nanocrystals are uncapped or uncoated, as described above. Uncapped and uncoated nanocrystals are free of relatively non-volatile organic capping (a monomolecular layer) or coating (a thicker than monomolecular layer) materials. Uncapped and uncoated nanocrystals can be used, for example, to form films comprising the nanocrystalline form that are substantially free of organic materials. Such films can be used in various electronic devices such as in dielectric layers, where the presence of organic contaminants is undesirable. Many art forms of uncapped and uncoated nanocrystals are prone to aggregation and clumping, and are difficult to disperse to provide substantially stable and homogeneous dispersions. However, uncapped and uncoated nanocrystalline forms of the disclosed subject matter, both in a dry form and in a dispersion in a liquid, are not prone to aggregation or clumping over a period of time, ranging from one hour to about six months. Uncapped and uncoated metal oxide nanocrystalline forms of the disclosed subject matter can be dispersed to form substantially stable and homogeneous dispersions particularly in polar organic solvents such as methanol, ethanol, or isopropanol, optionally containing various amounts of water. Such dispersions can be used to form films and coatings substantially free of organic contaminants, with any need to resort to high temperature techniques such as sintering, which can employ temperatures approaching 600° C. Such high temperatures can be incompatible with various materials and methods used in semiconductor fabrication and in other fabrication techniques. In certain semiconductor applications, organic contaminants provide undesirable electrical properties. Uncapped and uncoated nanocrystals of the disclosed subject matter can have a surface wetted with a volatile liquid such as an alcohol or water, wherein the volatile liquid can be removed if necessary without resorting to elevated temperatures, for example temperatures over 100° C., or can include nanocrystals with surface metal alkoxide or metal hydroxide groups, or a layer of a volatile solvent such as an alcohol or ether. The presence of a volatile alcohol or ether on a nanocrystal surface does not impair processing into organic contaminant-free materials.

Alternatively, the nanocrystalline forms of the present disclosure can be capped or coated, such as with an organic coating material. As is well known in the art, certain nanoparticles, such as gold nanoparticles, can be stabilized against aggregation by formation of a monomolecular capping layer around the nanoparticle, such as a layer composed of alkanethiol molecules wherein the sulfur and the gold interact non-covalently. Analogously stabilized metal oxide nanocrystals are termed capped nanocrystals herein, and can include organic coating materials on their surfaces. Organic coating materials typically include long chain organic molecules such as fatty acids, fatty alcohols, and fatty amines. When more than a single molecular layer is present, the nanocrystals are referred to as coated nanocrystals. A coated nanocrystal is necessarily also a capped nanocrystal, but a capped nanocrystal has only the molecular single layer. Nanocrystals that are capped or coated can be found to be stabilized against aggregation or clumping, and also can be more easily dispersed in certain types of liquids, such as non-polar organic solvents including aliphatic and aromatic hydrocarbons. Examples of organic coating materials include decanoic acid, oleic acid, oleylamine, and oleyl alcohol. Such coating materials are relatively non-volatile and would require high temperatures to remove. Coated nanocrystals can typically be more readily dispersed in non-polar organic solvents such as hexane or benzene than can uncapped and uncoated nanocrystals. Coated nanocrystals also can suffer no disadvantage in applications where an organic contaminant-free material is not needed, such as in a coating for metal, plastic, glass, skin, or the like. For example, a sunblock formulation wherein zinc oxide nanocrystals are dispersed in a carrier oil or cream base are not negatively affected by the presence of a capping or coating layer on the nanocrystals, and a capping or coating layer may be necessary to achieve good dispersion in the hydrophobic oil or cream. Additionally, supercritical carbon dioxide, generally considered to be equivalent to a non-polar organic solvent in solvating properties, can be a dispersant for nanocrystals, such as coated or capped nanocrystals.

Various embodiments of the disclosed subject matter are directed to methods to prepare nanocrystalline forms as described above. In an embodiment, a method of preparation of a metal oxide nanocrystalline form comprising contacting an metalorganic precursor, wherein the metalorganic precursor comprises a single metallic element or more than one metallic element, and a liquid substance comprising an alcohol at an elevated temperature of less than about 350° C., to provide the plurality of metal oxide nanocrystals having a narrow size distribution, is provided. The metalorganic precursor can be a metal alkoxide, a metal carboxylate, or a metal complex such as a metal acetoacetonate. Other metal salts and complexes can be used, such as a bulk metal oxide not in nanocrystalline form that can be dissolved in a suitable solvent. For example, in the preparation of barium titanate in a nanocrystalline form of the present disclosure, the barium can be provided in metalorganic form of an alkoxide, such as a benzoxide, that can be prepared by dissolving barium metal in benzyl alcohol. Alternatively, the barium can be provided in metalorganic form by dissolving bulk barium oxide in an alcohol such as ethanol or isopropanol. This can be mixed with a titanium alkoxide, such as titanium isopropoxide, to provide a bimetallic metalorganic precursor solution that can be heated in the presence of the liquid medium comprising an alcohol to provide a nanocrystalline form of the present disclosure.

The reaction mixture of the metalorganic precursor and the liquid substance comprising an alcohol is exposed to a temperature of less than about 350° C. For example, the reaction mixture can be exposed to a temperature of about 80-230° C. to provide a nanocrystalline form. The nanocrystalline form, which can be present as a stable dispersion in the reaction mixture medium, can be collected, such as by centrifugation, to provide the nanocrystalline form as a dry powder. Alternatively, it can be handled in that dispersion. To facilitate formation and collection of the nanocrystalline form, a reagent can be added after the step of heating. The reagent can amount to up to about 20% of a volume of the precursor solution. The reagent can include a polar organic solvent such as ethanol, or acetone. The reagent can facilitate recovery or collection of the nanocrystals from the dispersion, such as by centrifugation.

The metalorganic precursor can include only a single metallic element. Examples include titanium, zirconium, hafnium, vanadium, niobium, tantalum, tungsten, manganese, iron, cobalt, nickel, copper, zinc, gallium, indium, tin and cerium. For example, to prepare titanium oxide in a nanocrystalline form, the metalorganic precursor includes only titanium as a metallic element in salt or complex form. Other metalorganic precursors including only a single metallic element can include zinc and zirconium.

Alternatively, the metalorganic precursor can include two or more metallic elements. For example, the metalorganic precursor can include a first metallic element selected from the group consisting of titanium, zirconium, hafnium, vanadium, niobium, tantalum, tungsten, manganese, iron, cobalt, nickel, copper, zinc, gallium, indium, tin and cerium, and a second metallic element selected from the group consisting of barium, strontium, calcium, lithium, lead, yttrium, bismuth, lanthanum, a rare earth metal, titanium, zirconium, iron, copper, manganese, cerium, and cobalt. More specifically, to prepare a nanocrystalline form of barium titanate, the metalorganic precursor comprises barium and titanium salts or complexes. Examples include a titanium alkoxide, titanium acetate, or titanium acetoacetonate and a barium alkoxide, barium acetate, or barium acetoacetonate.

For example, an embodiment of the disclosed subject matter concerns a method for the preparation of ligand surface-capped nanocrystals of BaTiO₃, comprising: dissolving barium metal in an alcohol to provide a barium alcoholate solution; then, contacting the barium alcoholate solution with a solution of a titanium alkoxide to provide a bimetallic precursor solution; then, contacting the bimetallic precursor solution at an elevated temperature for a period of time comprising at least about 24 hours with a solvent/ligand mixture; then, adding a polar solvent to precipitate the nanocrystals; and lastly, separating the ligand surface-capped nanocrystals from the polar solvent.

In a method, the elevated temperature can be about 80° C. to about 230° C. Due to the presence of volatile alcohols, a temperature at the higher end of this range would be above their boiling point, so the reaction can be carried out under self-generated pressure in a pressure containment vessel. For example, a pressure of about 20 atm to about 30 atm can be used.

Optionally, the reaction mixture can include an organic coating material such that a capped or coated nanocrystalline form is obtained. The organic coating material can include an alkanoic acid, a saturated or unsaturated fatty acid, decanoic acid, oleic acid, an alkylamine, a fatty amine, oleylamine, an alkanol, a fatty alcohol, or oleyl alcohol, or a combination thereof.

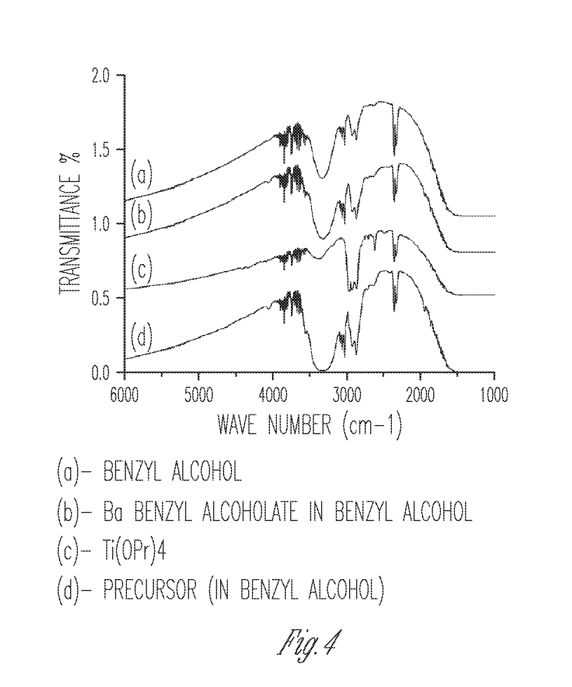

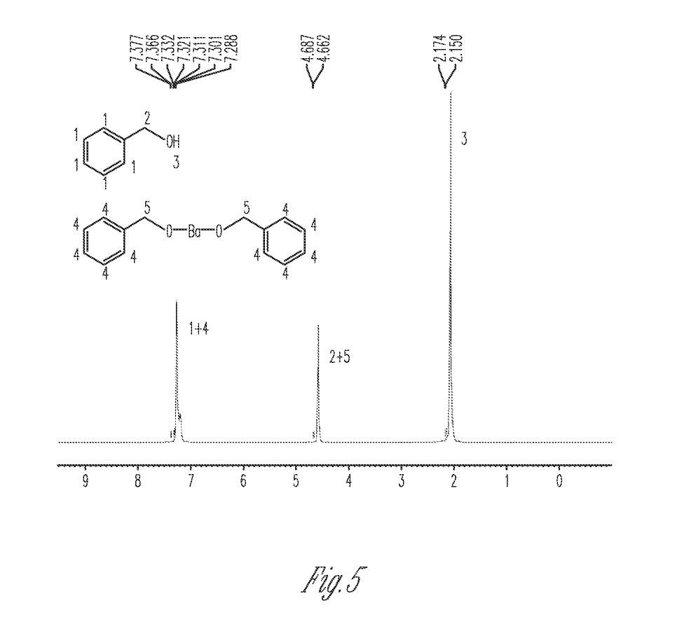

For example, in the preparation of nanocrystalline forms of barium titanate, variations on the method have been investigated, as described below. The reaction mechanism leading to the formation of BaTiO₃ is believed, while not wishing to be bound by theory, to proceed mainly via a pathway involving C—C bond formation between the alcohol and the isopropanolate of the titanium alkoxide. This mechanism can proceed under ambient pressure in oleylamine. See Examples 1-4: benzyl alcohol (BzOH) in Cases I and II, mixture of BzOH and oleyl alcohol (OLOH) in Case III, and OLOH in Case IV concerning use of a bimetallic metalorganic precursor. The precise nature of this bimetallic precursor is yet to be determined but IR and NMR studies suggest it is not a simple mixture of alkoxides. The color change in the synthesis of the precursor (from clear to white precipitate solution) also supports this hypothesis. IR and NMR studies of the precursor in case I were performed (see FIGS. 4 and 5). From the IR and NMR results it is clear that the titanium isopropoxide undergoes alcoholysis to form a precursor. According to the proposed reaction mechanism, titanium isopropoxide reacts with the benzyl alcohol via the formation of a C—C bond through several steps to form the organic species with a Ti—O—Ti bond such as shown below.

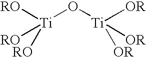

Then it is believed, in a later stage, the Ba benzyl alcoholate reacts with this Ti—O—Ti species to form BaTiO₃ nanoparticles by the Ba²⁺ substituting the isopropanol groups. Since the synthesis of the precursor is in the very early stage of the reaction, the precursor is probably a mixture of the Ti—O—Ti species (above) or some intermediate product before forming the Ti—O—Ti species, Ba benzyl alcoholate, and extra benzyl alcohol.

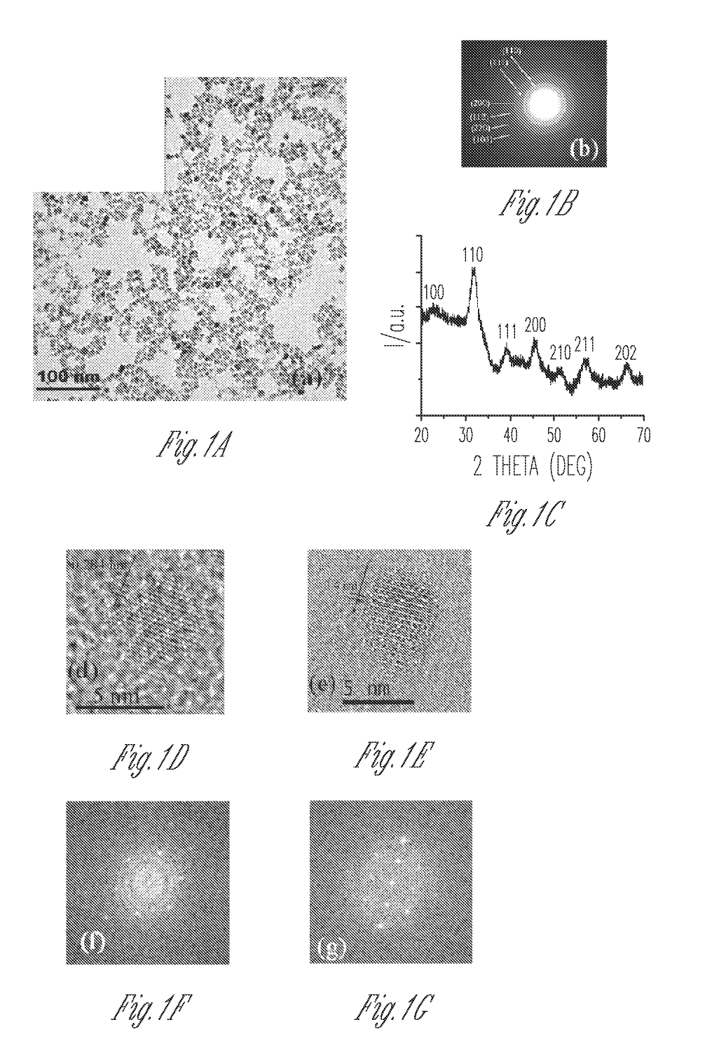

FIG. 1 shows the TEM and XRD characterizations of 6-10 nm BaTiO₃ nanocrystals capped with decanoic acid (case I). XRD and Selected Area Electron Diffraction (SAED) patterns are shown in FIGS. 1(b) and 1(c) where BaTiO₃ phase is assigned to the crystal structure and crystallinity is confirmed. An overview TEM image (FIG. 1(a)) at low magnification shows non-aggregated BaTiO₃ nanocrystals with average diameter 6-10 nm without any presence of larger particles or agglomerates. HRTEM patterns of individual nanocrystals are shown in FIGS. 1(d) and 1(e). In FIG. 1(d), the nanocrystal is oriented in its <111> zone axis and the corresponding power spectrum (PS) is shown in FIG. 1(f). In FIG. 1(e), the zone axis of the nanocrystal is <100> and the corresponding PS is shown in FIG. 1(g). HRTEM studies and PS (FIGS. 1(d) to 1(g)) provide extra evidence that the particles are well crystallized.

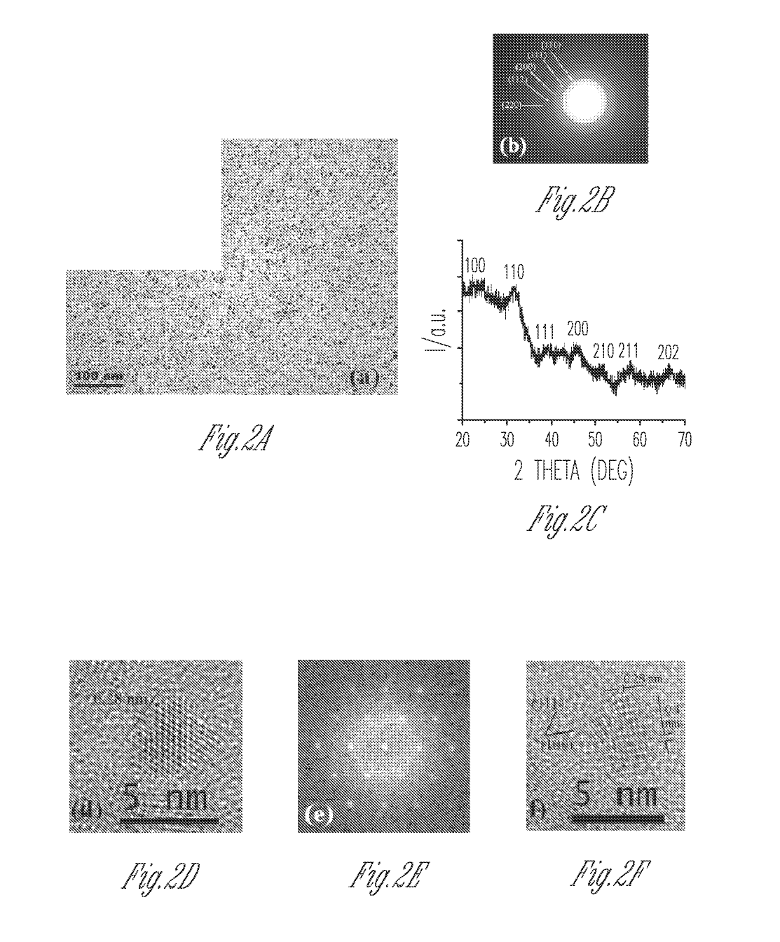

FIG. 2 shows the TEM and XRD characterizations of 3-5 nm BaTiO₃ nanocrystals capped with oleyl acid (case II). Combining the technique of XRD and SAED (FIGS. 2(b) and 2(c)), BaTiO₃ phase is assigned to the crystal structure, even though diffraction peaks in the XRD pattern (FIG. 2(c)) are broadened substantially due to the small crystal size and the organic coating. The low angle background is attributed to the small crystal size, extra organic coatings and solvents, and the possible effect of the glass substrate when the XRD experiment was carried out. An overview TEM image (FIG. 2(a)) at low magnification shows non-aggregated BaTiO₃ nanocrystals with average diameter 3-5 nm without any presence of larger particles or agglomerates. HRTEM patterns of individual nanocrystals are shown in FIGS. 2(d) and 2(f). In FIG. 2(d), a representative nanocrystal is oriented in its <111> zone axis and the corresponding power spectrum (PS) is shown in FIG. 2(e). Although most of the 3-5 nm BaTiO₃ nanocrystals have a single domain, twinning by sharing one of the (111) planes is also observed and one example is shown in FIG. 2(f). The percentage of the BaTiO₃ nanocrystals with twinning is observed to be around 5% under TEM. SAED, HRTEM studies and PS (FIGS. 2(b), and 2(d) to 2(f)) provide extra evidence that the particles are well crystallized. Compared with case I (FIG. 1), the crystal size is decreased which is attributed to the fact that oleic acid acts as a longer chain carboxylic acid and a stronger coordinating ligand than decanoic acid.

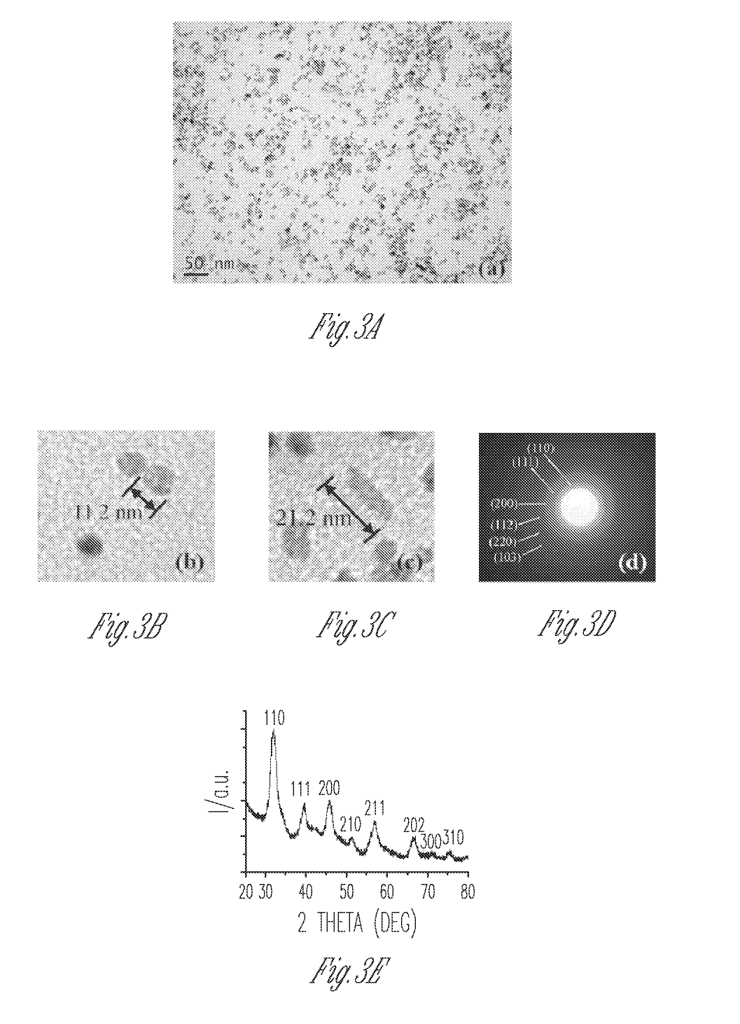

Bigger BaTiO₃ nanocrystals in the size range 10-20 nm with a more regular morphology than cases I and II can be obtained when employing oleyl alcohol (OLOH) with the use of benzyl alcohol (BzOH) in the precursor synthesis (case III, FIG. 3). Again the BaTiO₃ phase is assigned to the crystal structure, FIGS. 3(e) and 3(d)) and good ability to disperse into hexane is found (FIG. 3(a)). The low angle background is attributed to the small crystal size, extra organic coatings and solvents, and the possible effect of the glass substrate. Interestingly, the nanocrystals synthesized in case III display different morphologies. Most of these nanocrystals are spherical (FIG. 3(b)), but cubic, elliptical, triangular and rod (FIG. 3(c)) shapes are also observed. The sizes of these particles are around 10-12 nm and the size of these nanorods is around 20 nm in length and 6 nm in width. By employing only OLOH (case IV), nanocrystals with a much smaller size (2-3 nm) are found. We attribute the lack of morphology control in case I and II to the behavior of BzOH during the first stage of the synthesis (precursor synthesis). Since OLOH is a much stronger coordinating alcohol than BzOH, replacing BzOH by a mixture of BzOH and OLOH (case III) or only OLOH (case IV) in the precursor synthesis improves homogenous nucleation and therefore improves crystal morphologies. The much smaller crystal size in case IV (compared to case III) is attributed to the fact that OLOH is a longer chain alcohol than BzOH and dissolving Ba in only OLOH without BzOH results in much more oleyl groups binding onto nanocrystal surfaces.

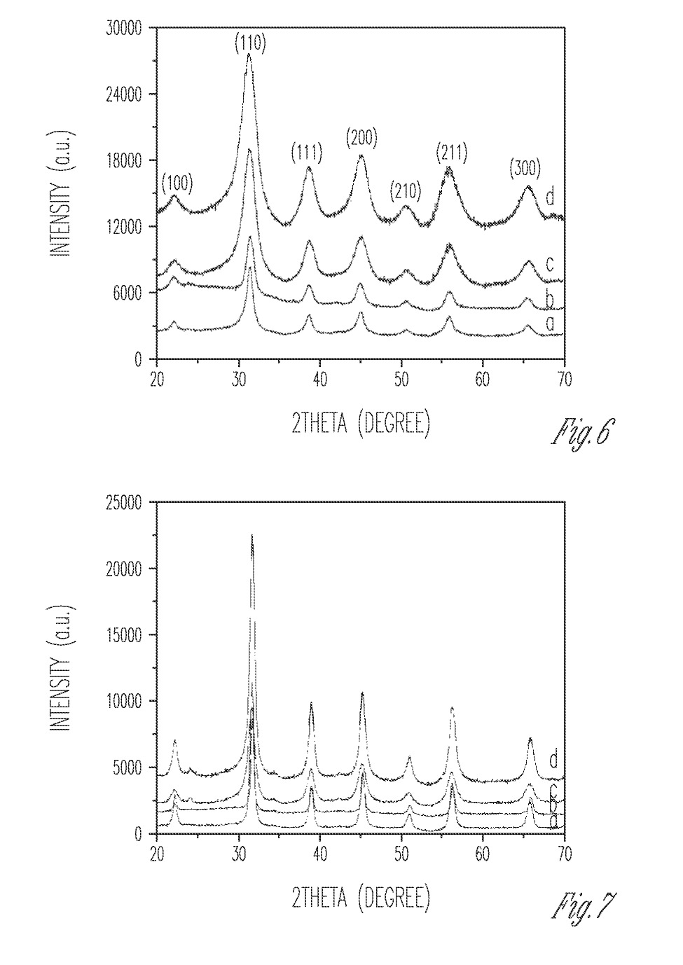

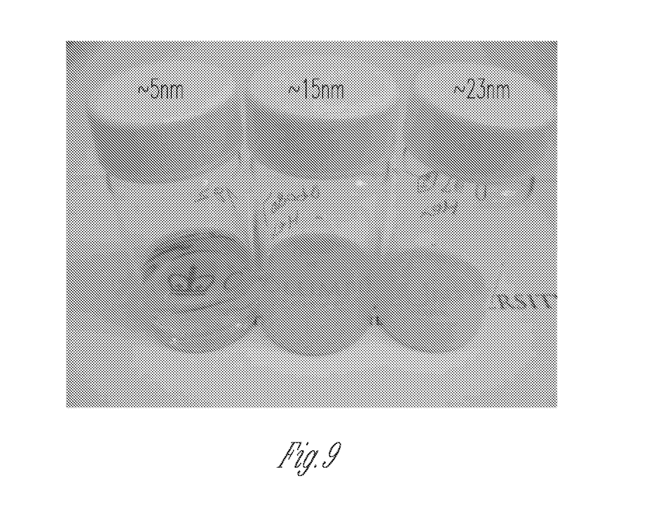

FIG. 6 shows the typical X-ray diffraction (XRD) patterns of the samples prepared in different alcohol or alcohol mixture using barium and titanium isopropoxide as a precursor. All diffraction peaks can be assigned to the BaTiO₃ phase (JCPDS No. 31-174) without any indication of crystalline byproducts such as BaCO₃ or TiO₂. The measurement indicates the exclusive presence of a perovskite BaTiO₃ phase in high crystallinity and high purity. The broad diffraction peaks suggest small crystalline sizes on the nanometer scale. According to the degree of peak broadening, the crystal sizes decrease in the following order of solvents: ethanol, 95% ethanol> mixed ethanol and isopropanol> isopropanol, which is consistent with the TEM observation. The TEM images (FIG. 8) indicate that the samples consist of individual BaTiO₃ nanocrystals with narrow size distribution and no aggregation, except for that prepared in isopropanol, which show irregular in shape and some extent of aggregation of smaller nanocrystals. Interestingly, BaTiO₃ nanocrystals show more regular and uniform when prepared in mixed ethanol and isopropanol (FIG. 8d) than prepared in sole alcohols (FIGS. 8a and 8b). Both XRD and TEM results indicate that the BaTiO₃ crystal sizes can be tunable with different alcohols as solvents. Besides, the crystal size can vary with temperature, and crystallization time, but the variation is not as obvious as that in different solvents. In addition, the diffraction peak in the 2 theta region of 40-50° is usually characteristic for the presence of either cubic or tetragonal BaTiO₃ structure. However, the diffraction peaks are too broad to discriminate between the cubic and tetragonal BaTiO₃ structures due to the small particle sizes.

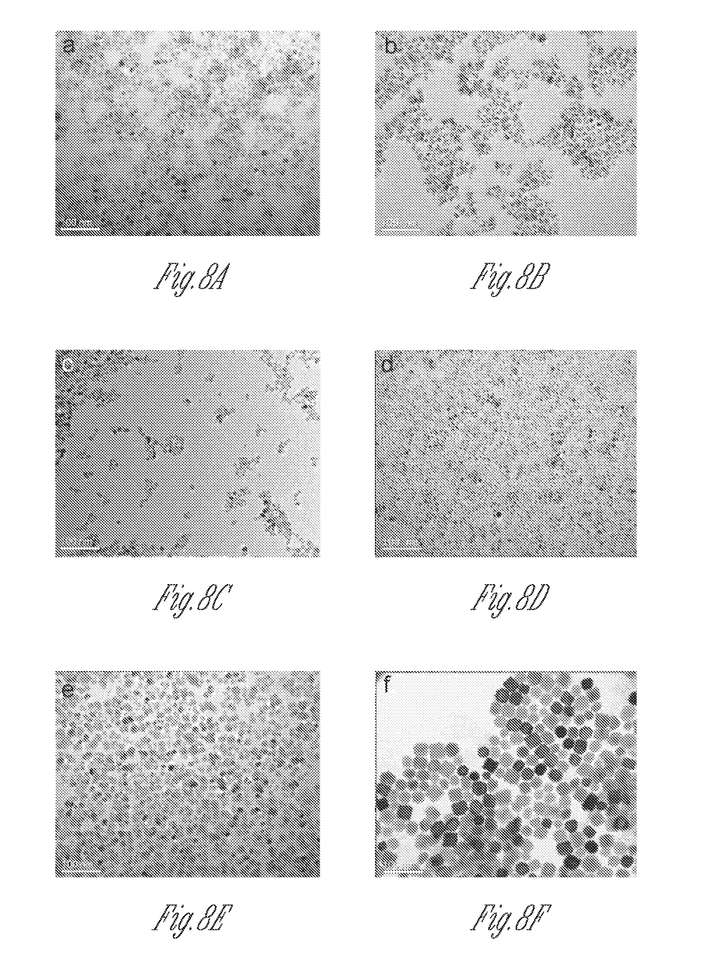

FIG. 7 shows the XRD patterns of the samples prepared using BaTi ethylhexano-isoproxide as a source. Likewise, the measurement indicates the presence of the perovskite BaTiO₃ phase in high crystallinity and high purity. Very trace amount of BaCO₃ could be detected for the samples prepared in ethanol and isopropanol, but could totally vanish when extending the duration time from 48 hrs to 72 hrs. The diffraction peaks appear sharper as compared with those prepared using metal Ba and titanium isopropoxide as a precursor, showing the formation of larger nanocrystals when using BaTi ethylhexano-isoproxide as a source. This is also confirmed by the TEM results in FIG. 8 (FIG. 8e vs FIG. 8b, FIG. 8f vs FIG. 8c). Moreover, when 95% ethanol is used as the solvent, the sizes of nanocrystals increase from ˜15 nm to ˜25 nm, indicating that trace amount of water in ethanol may tune the alcoholysis rate and therefore the nucleation process. The TEM image (FIG. 8f) shows the existence of individual BaTiO₃ nanocrystals in regular shape (most of them are cubic and spherical). In addition, the crystal size can further increase (up to ˜100 nm) by increasing the volume ratio of 95% ethanol/BaTi ethylhexano-isoproxide (1:1-10:1), while small amount of amorphous phase (tiny and irregular phase) may co-exist as well. The addition of alkali (such as potassium hydroxide (KOH), or tetrapropylammonium hydroxide (TPAOH)) in a ratio of OH/Ba=½ can remove the amorphous phase and reduce the crystal size.

From the above results, one can conclude that the simple solvothermal process of a BaTi metalorganic source in an alcohol solvent is a versatile method for producing highly crystalline and aggregate-free BaTiO₃ nanocrystals with variable sizes and narrow size distribution. The tunable nanocrystal sizes (5-100 nm) can be realized by using different alcohol solvents and BaTi metalorganic sources, which provide tunable rates of alcoholysis process and further nucleation of BaTiO₃ nanocrystals.

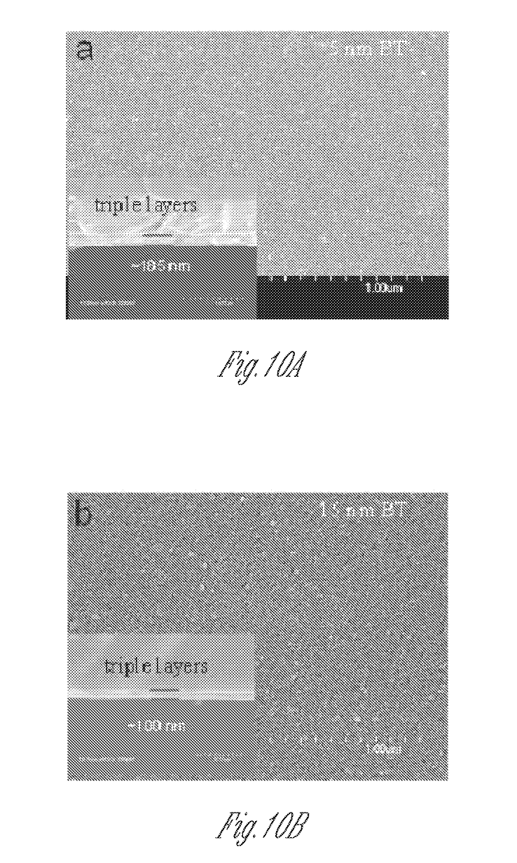

The aggregate-free nature of BaTiO₃ nanocrystals enables easy modification of the crystal surface with a variety of surface capping agents (e.g. oleic acid), surfactants, or polymers. For instance, a simple solution processing between as-precipitated BaTiO₃ nanocrystals (wet) and oleic acid at elevated temperature (80° C.) can provide strong oleic acid binding to the nanocrystal surface. The oleic acid-coated BaTiO₃ nanocrystals are highly dissolved in non-polar solvents such as hexane and toluene to obtain homogeneous and transparent/semitransparent suspensions (FIG. 9). Moreover, the BaTiO₃ nanocrystals synthesized in 95% ethanol show higher solubility and stability in polar solvents such as ethanol than those synthesized in anhydrous ethanol, probably because of more surface hydroxyl groups and enhanced surface polarity for the crystals prepared in trace amount of water. The suspension can be stable for weeks without precipitation. Only a small number of nanoparticles may be precipitated out in a month, but they can be easily re-dispersed to ethanol using a simple sonication process.

In various embodiments of the present disclosed material, a substantially homogeneous dispersion of the nanocrystalline form or a substantially homogeneous dispersion of a nanocrystalline form prepared by a disclosed method, in a liquid, are provided. The nanocrystals of the dispersed nanocrystalline form can be uncoated and uncapped, or can be capped or coated, as described above. The liquid can comprise a non-polar organic solvent or a polar organic solvent or a mixture thereof. The liquid can comprise water, or a mixture of water with a water-soluble organic solvent. As discussed above, when the nanocrystalline form is capped or coated, dispersions can be readily formed in non-polar organic solvent such as hexane or toluene, or a mixture thereof. When the nanocrystalline form is uncapped and uncoated the liquid can be a polar organic solvent such as ethanol or methanol, or a mixture thereof.

The dispersion can be substantially physically stable over a period of time under normal gravitation, such that substantial amounts of precipitation of the nanocrystalline form does not occur over a period of time, which can range from about one hour to about six months. The dispersion can also be substantially chemically stable, wherein decomposition of the nanocrystalline form to another chemical entity in the presence of the liquid does not take place to any great extent.

A substantially homogeneous dispersion can include additional ingredients. For example, a dispersion can further include a surfactant, a polymer, a liquid crystal forming material, a phospholipid, or a mixture thereof.

Additionally, a dispersion can include a matrix precursor, that is, a material adapted to form a matrix for the nanocrystalline form, such as upon drying or removal of the liquid substance in formation of a film comprising the nanocrystalline form. A matrix can serve to provide physical cohesiveness to such a film, or to alter the properties of the film, or both. A matrix precursor can be an organic matrix precursor adapted for polymerization for formation of an organic matrix, or an inorganic matrix precursor adapted for formation of an inorganic matrix, or a mixed organic/inorganic matrix precursor adapted for formation of an organic/inorganic matrix, or any mixture thereof. An example of an organic matrix is an organic polymer, such as a poly(methyl methacrylate), a polyurethane, or a block organic copolymer. An example of an inorganic matrix is amorphous silica. An example of an organic/inorganic matrix is an amorphous silica modified with oligomeric or polymeric organic domains. For example, an organic/inorganic matrix can be composed of interpenetrating networks of silica and an organic polymer, or can be composed of a block copolymer comprising organic and inorganic domains. The inorganic domain can be amorphous silica.

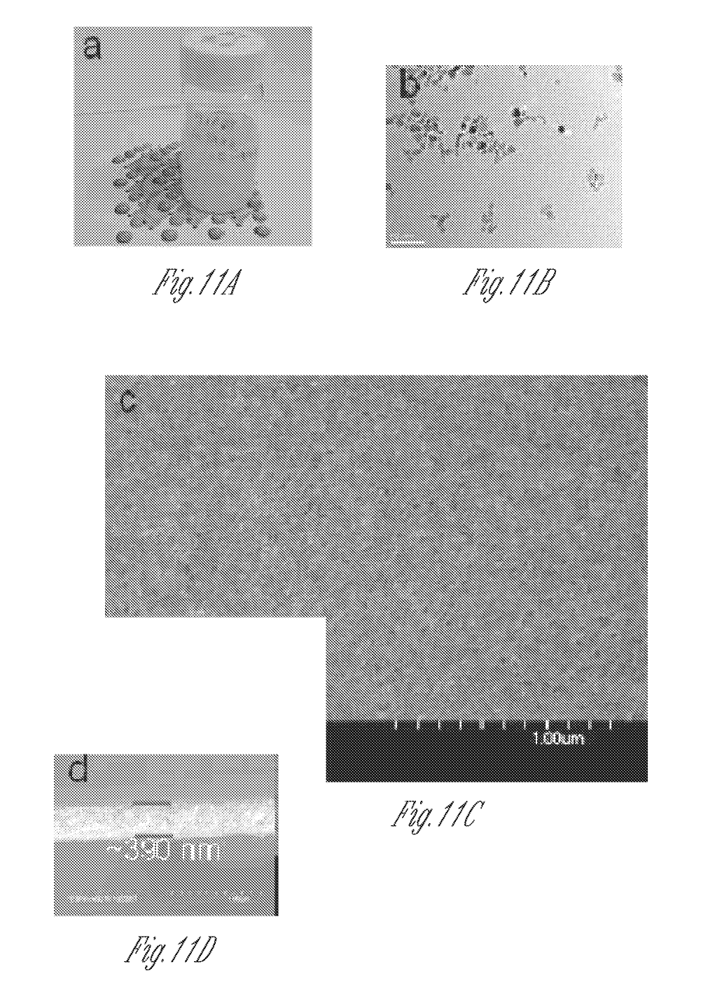

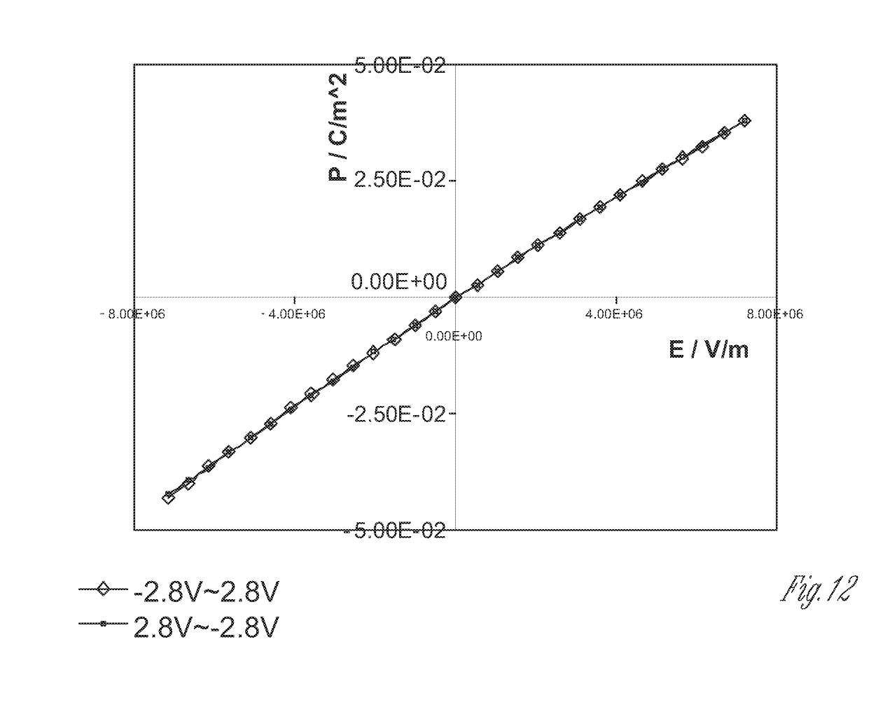

The stable and homogeneous nanocrystal suspension offers advantages in thin film processing on both solid substrates (e.g. Si wafer) or flexible substrates (e.g. plastic) by using coating (spin-coating, dip-coating, and cast-coating) techniques. Uniform, dense and crack-free BaTiO₃ thin films (50-500 nm in thickness) were prepared by multiple spin-coating of the nanocrystal suspension (either hexane suspension of oleic acid capped nanocrystals or ethanol suspension of uncoated nanocrystals). A thermal treatment at 350-400° C. was applied to remove the surface oleic acid coatings (FIG. 10). For the BaTiO₃ thin films prepared from the uncoated BaTiO₃ nanocrystals, low temperature baking at 60° C. is what is necessary to remove the ethanol residual (FIG. 11) and stabilize the thin films. Dielectric constant measurement shows a high k value of ˜750 for the BaTiO₃ nanocrystal (˜8 nm crystal size) thin film while no significant hysteresis loop can be observed in the polarization vs electric field plot (FIG. 12), suggesting that there are no ferroelectric domains in the nanocrystals smaller than 10 nm in diameter. The low temperature process also allows the thin film fabrication on other flexible substrates (e.g. plastic substrate) for flexible electronics application. The BaTiO₃ nanocrystal thin films are optically transparent in the visible light range because of the nanosized crystals and good film uniformity.

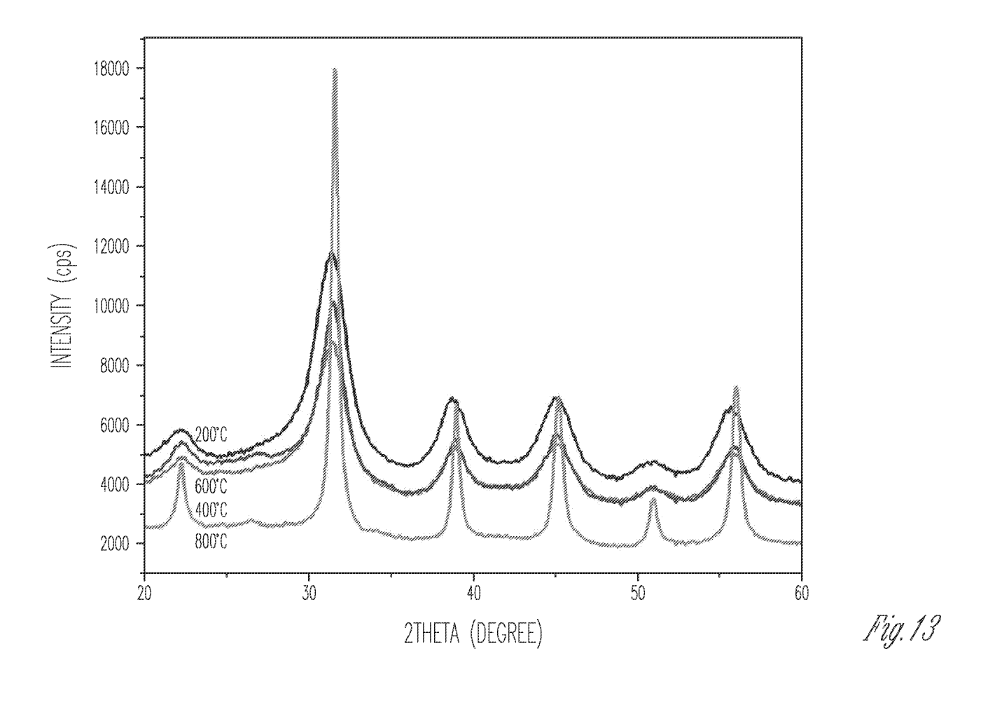

The thin films also show high thermal stability. The XRD results show they can be stable up to 500° C. with no sign of crystal growth, and only at higher temperature (800° C.) one can see significant crystal merge and growth in the thin films, according to the extent of diffraction peak sharpening during the thermal treatment at various temperatures (FIG. 13).

The oleic acid-coated BaTiO₃ nanocrystals also enable an easy incorporation with other media (e.g. polymers, liquid crystals, etc.), which is favorable for BaTiO₃-based nanocomposite thin film processing and can further improve the basic mesogenic properties of nematic liquid crystals (such as higher clearing temperature, larger birefringence and enhanced dielectric response) when doped at low concentration (<1 wt %).

Accordingly, various embodiments of the disclosed subject matter include methods of forming a film comprising a plurality of metal oxide nanocrystals, the method comprising disposing a dispersion of the nanocrystalline form or a dispersion of a nanocrystalline form prepared by a method of the disclosed subject matter, or disposing the dispersion of the plurality of nanocrystals, or disposing a dispersion prepared by a method of the disclosed subject matter, on a substrate, then, at least partially removing the liquid substance, and the reagent if present, or the liquid from a dispersion therein, to provide the film disposed on the substrate.

A matrix precursor can be included in the dispersion of nanocrystals that is disposed on the substrate such that the matrix precursor can be polymerized to provide the film disposed on the substrate upon removal of liquid materials, wherein the film comprises a matrix. As described above, the matrix precursor can be organic, inorganic or organic/inorganic, and can be polymerized to provide a respective matrix composed of an organic, inorganic, or organic/inorganic material. The film formed by removal of the liquid substance thus includes the matrix material in which the plurality of nanocrystals is dispersed or embedded. The matrix formed by polymerization of the matrix precursor can add film strength for greater facility of processing.

The substrate upon which the dispersion is disposed and upon which the film is formed by removal of the liquid substance can be an electrically insulating, conductive or semi-conductive material. The substrate can be a flat, curved, or irregular surface, which can be composed of a solid material comprising silicon, silicon nitride, silica, diamond, or an organic plastic.

Alternatively, the substrate can be a surface that is to be coated by the dispersion, such as human skin. For example, a plurality of zinc oxide nanocrystals, optionally including a matrix material such as a cream, can be disposed on human skin to form a sunscreen protective coating. In similar ways, other materials, for example metal or glass, can be coated with a protective layer.

The dispersion can be disposed on the substrate by a process comprising spin-coating, dip-coating, cast-coating, printing, or spraying, as are well known in the art. The stability of the dispersion, including a dispersion of uncapped and uncoated metal oxide nanocrystals, allows the dispersion to be handled over a period of time without greatly changing in solids content through precipitation of significant portions of the dispersed nanocrystalline solid metal oxide. The liquid substance, and reagent if present, or the liquid if a dispersion therein is used, can be removed from the dispersion, for example by volatilization, leaving the film as a residue upon the substrate surface. For example, liquid materials can be removed at least in part by evaporation or volatilization, or evaporation under a vacuum, or through the application of heat, or any combination thereof.

The film can be further processed in substantially dry form on a suitably adapted substrate by heating or sintering the film at any suitable temperature. For example, sintering can be carried out at up to 600° C., although lower temperatures can also be used. The substrate is adapted to be stable at a sintering temperature selected. When the film comprises, for example, a plurality of uncapped and uncoated nanocrystals, lacking a matrix precursor, or with a matrix precursor that forms a heat-stable inorganic material such as silica, a film substantially free of organic material can be obtained without any need to resort to high sintering temperatures to remove organic residues. Organic residues can be undesirable; the residues can, for example, degrade electrical properties of the film. The film can also be substantially free of voids. For example, the nanocrystals can be in direct contact with each other such that a continuous film is obtained and, when a matrix is present, the matrix can also serve to make the film continuous and substantially lacking in voids and defects. Alternatively, the film can be adapted to have a particular proportion of voids; for example the film can be adapted to have 10% voids, or 20% voids. The relative proportion of the voids to the film can be controlled or tuned.

The film can have a thickness of about 10 nm to about 1 millimeter. The film can be formed of substantially a single layer of nanocrystals, or can include many layers of nanocrystals in the thickness dimension. The film can be of substantially any length and breadth, depending on the dimensions of the substrate on which it is disposed. The film can have a high dielectric constant or dielectric strength. A high dielectric constant or strength can be a dielectric constant greater than ten. In various embodiments, the film can have a dielectric constant of about 80 to about 750.

The film of the disclosed subject matter can be composed of a single composition of nanocrystalline form, which can be embedded or disposed in a matrix material, or which can not include a matrix material. An example of a single type of nanocrystalline form is barium titanate. The film can include only the nanocrystalline form having a single component wherein substantially all the nanocrystals are of approximately the same dimension, having a narrow size distribution, or a monodisperse size distribution. Alternatively, the film can include nanocrystals of a single chemical composition, for example barium titanate, but include a mixture of different sizes of the nanocrystals, for example a mixture of barium titanate nanocrystals of two different size profiles, for example a 3-5 nm set and a 10-20 nm set. Or, the film can include more than one type of nanocrystal including a single type of metal oxide, but wherein some nanocrystals are uncapped and uncoated and others in the same film are capped or coated.

An example is a film composed of barium titanate and silica, which can be prepared from a dispersion of barium titanate and a matrix precursor including a tetralkylorthosilicate. The liquid can be ethanol, such as 95% aqueous ethanol (azeotrope ethanol).

Alternatively, the film can include nanocrystals having compositions including different chemical compositions. For example, a film can include barium titanate crystals, and lead titanate crystals.

The film can further include a block co-polymer, such as a block polyethylene-polypropylene or a block copolymer surfactant such as a Pluronic® surfactant, or triblock copolymer surfactant such as P₁₂₃ of the Pluronic family.

The film can further include an organic component used in generation of a mesoporous material. As described above, a mesoporous material is a micro-structured material that can be prepared by hydrolysis and condensation of tetraethyl orthosilicate templated by supermolecular arrays of surfactant (micellar rods). After the organic-inorganic composites are formed, the organic templates can be removed by thermal treatment or solvent extracting, leaving behind an inorganic matrix with ordered pores and channels corresponding to the original surfactant supermolecular templates.

The organic compound used in generation of a mesoporous material can include MCM-41, MCM-48, which are products originally developed by researchers from former Mobil Company, and SBA-15, or SBA-16, products developed by researchers from University of California Santa Barbara, as are well known in the art.

Such films can also be deposited on substrates, which can be flat, curved, or irregular, and can be formed of materials that include silicon, silicon nitride, silica, diamond, or an organic plastic.

The film, comprising a particular type or types of the nanocrystalline form can substantially retain properties of the respective type of nanocrystalline form, that is, formation of a film or coating of the nanocrystalline form does not eliminate or substantially alter the unique properties that can result from the material being in the nanocrystalline form, as opposed to a standard bulk form not including nanocrystals. An example is electrical properties such as dielectric constant. Other properties of the nanocrystalline form that can be substantially retained by a film formed of the nanocrystals include density properties, spectral properties such as absorption maxima, extinction coefficients, reflectivity parameters, luminescence, or any combination thereof. The film can substantially retain hardness properties or scratch resistance properties of the nanocrystalline form, or can retain thermal properties of the nanocrystalline form Again, the film can include a nanocrystalline form wherein the nanocrystals are uncapped and uncoated, enabling the formation of organic contaminant free films. The absence of the organic contaminants can serve to preserve the properties of the nanocrystalline form in the film.

Similarly, a coating layer can be formed including the nanocrystalline form disclosed and claimed herein, or a nanocrystalline form prepared by a method disclosed and claimed herein. Alternatively, a coating layer can be formed using the dispersion disclosed and claimed herein, or a dispersion prepared by a method disclosed and claimed herein. For example, a coating layer can include an embodiment of the nanocrystalline form of zinc oxide, titanium oxide, or zirconium oxide. A coating layer can further include a monomer adapted for polymerization, or a polymer, or both.

A coating layer as disclosed and claimed herein can be adapted for application to human skin. For example, the nanocrystalline form can include zinc oxide, and the coating layer can include a cream or oil in which the nanocrystalline form is dispersed, that can be applied to human skin to prevent sunburn. Alternatively, the coating layer can be adapted for application to a metal, plastic, or glass surface. For example, the coating layer can include titanium oxide and be adapted for application to a glass surface to alter the transmission or reflection properties of the glass, or to provide a solar self-cleaning glass surface, or both. The coating layer can include a suitable organic, inorganic, or organic/inorganic matrix adapted to provide adhesion to the surface. For example, a metal surface such as an automobile body can be covered with the coating layer as disclosed and claimed herein, wherein a nanocrystalline form of titanium or zirconium oxide is dispersed in an organic polymer adapted to adhere to a metal surface, to provide an automobile paint.

In various embodiments in accordance with the teachings described herein, nanoparticles, may be used in various electronic and/or electro-optic apparatus. Such nanoparticles may include metal oxide nanoparticles having high k dielectric constants, that is, having dielectric constants of ten or greater. A mechanically and thermally stable thin film of such metal oxide nanoparticles between 10-1000 nm in thickness (or thicker) can be generated on substrates. A film of metal oxide nanoparticles may be referred to as a high k film, that is, a film have a high dielectric constant k. A high dielectric constant can be a dielectric constant greater than about 10. In various embodiments, the film can have a dielectric constant of about 80 to about 750. The high k film may include one or more types of nanoparticles. The nanoparticles in the high k film may be amorphous nanoparticles, crystalline nanoparticles, or a combination of amorphous nanoparticles and crystalline nanoparticles. In addition, the nanoparticles may be capped or uncapped. The substrates, on which the high k film may be disposed, may include Si, SiO₂, SiN, silicon-on-insulator, or other wafer known to the semiconductor industry.

Such thin, high k films can be used in the manufacture of ultracapacitors. As discussed above, ultracapacitors offer very high charge densities and short charge/discharge times, plus a very high capacity for recharge without degradation. As discussed above, ultracapacitors make use of very thin films, which can be on the order of nanometers, of high k materials, such as films prepared from the nanocrystalline forms herein. In an electrical double layer, the effective thickness of the “dielectric” of an ultracapacitor is exceedingly thin—on the order of nanometers—and that, combined with the very large surface area, is responsible for their extraordinarily high capacitances in practical sizes.

The nanoparticles of the high k film include a metal oxide of one of the many specified compositions stated previously in this disclosure. The various metal oxide compositions for inclusion in the high k film may include binary metal oxides such as, but are not limited to, titanium oxide (TiO₂), zirconium oxide (ZrO₂), hafnium oxide (HfO₂), vanadium oxide (V₂O₃), niobium oxide (Nb₂O₅), tantalum oxide (Ta₂O₅), tungsten oxide (WOₓ manganese oxide (Mn₃O₄), iron oxide (Fe₃O₄), cobalt oxide (CoO), nickel oxide (NiO), copper oxide (CuO), zinc oxide (ZnO), (cobalt, manganese)-doped zinc oxide, gallium oxide (Ga₂O₃), indium oxide (In₂O₃), tin oxide (SnO₂), ceria (CeO₂), and combinations thereof. The various metal oxide compositions for inclusion in the high k film may include perovskite ABO₃ structures such as, but are not limited to, barium titanate (BaTiO₃), strontium titanate (SrTiO₃), calcium titanate (CaTiO₃), barium strontium titanate ((Ba,Sr)TiO₃), barium lanthanum titanate ((Ba₁₋ₓLaₓ)TiO₃), lithium lanthanum titanate ((LiLaTiO₃), lead titanate (PbTiO₃), lead zirconium titanate (Pb(Zr,Ti)O₃), barium zirconate (BaZrO₃), lead zirconate (PbZrO₃), yttrium ferrite (YFeO₃), bismuth ferrite (BiFeO₃), yttrium barium copper oxide (YBCO), lanthanum manganese oxide (LaMnO₃), strontium cerium oxide (SrCeO₃), rare earth cobalt oxide (RECoO₃), and combinations thereof. The various metal oxide compositions for inclusion in the high k film may include other complex metal oxides such as, but are not limited to, indium tin oxide (ITO), lithium niobium oxide (LiNbO₃), garnet such as Bi₃Fe₅O₁₂, Y₃Fe₅O₁₂, and combinations thereof.

The metal oxides may be synthesized as metal oxide nanocrystals using a solvothermal approach. In an embodiment, a synthesis approach is based on the solvothermal reaction of a metal oxide precursor (such as metal alkoxides or metal acetylacetonates, or metal acetates) with an alcohol (ethanol, isopropanol, or oleyl alcohol), or the alcohol mixture (e.g. ethanol and isopropanol) or the alcohol with controlled amount of water (0-20 wt % water, e.g. 95% ethanol, azeotropic liquids) at a relatively low temperature (80-230° C.).

In various embodiments, suspensions are used where the suspension may be integrated into mixtures containing other components such as organic or inorganic materials. Such suspensions are comparable to solutions, in which the nanoparticle colloid is dispersed into the liquid such that it is similar in properties to a solution. Solutions are stable for prolonged periods of time without change. Processing temperature for generating the oxide nanoparticles may be relatively low, typically less than 250° C. Such processes may produce partially crystallized or amorphous materials that require further thermal treatment, which may lead to a loss of surface coating, aggregation, or precipitation from solution. The metal oxide nanoparticles are initially in the form of a suspension. For example, in a single processing step in ethanol under elevated pressure and temperature may be performed that results in virtually 100% crystalline barium titanate with high purity.

The metal oxide nanoparticle suspension may be mixed with another suspension to create a mixture. The second suspension contains a precursor that will allow the generation of a matrix, which may be referred to as a matrix precursor suspension. The matrix precursor suspension may be a precursor for the generation of a matrix composed of one or more of several materials. First, it may be organic (e.g. a polymer, such as PMMA or block copolymer) or inorganic (e.g. another metal oxide or SiO₂) or an organic-inorganic framework containing both block copolymer and SiO₂. The mixture of the nanoparticle oxide suspension and the matrix precursor suspensions can be applied onto a substrate in order to create a wet film that can be dried and or thermally processed to create a thin film. Such a process may be broadly referred to as a chemical deposition. The thin film may be patterned and processed using conventional semiconductor processing techniques to form the thin film as a component of an electric device in an integrated circuit.

The nanoparticles may be capped with ligands or not capped. The choice of capping or not capping can affect the solvent conditions that may be used in the processing. For example, a suspension mixture that utlilizes non-polar solvents is associated with nanoparticles and non-polar capping groups. An example of an idealized mixture may include BaTiO₃ nanoparticles suspended in ethanol with no ligand capping to be mixed with tetra-ethyl-ortho-silicate, or equivalently tetra-ethoxy-silane, (TEOS), a silica precursor such as, Si(OCH₂CH₃)₄, also in ethanol. An example of an idealized mixture may include BaTiO₃ nanoparticles suspended in ethanol with no ligand capping to be mixed with TEOS and a block co-polymer, such as block polyethylene-polypropylene (Pluronic surfactant e.g. P₁₂₃), a silica precursor (Si(OCH₂CH₃)₄) also in ethanol. An example of an idealized mixture may include BaTiO₃ nanoparticles suspended in ethanol with no ligand capping to be mixed with a silica precursor and organic component used in the generation of a mesoporous material, such as MCM-41, MCM-48, SBA-15, SBA-16, etc. In each example, identical solvent suspension compatibility can generate a homogeneous mixture suitable for chemical deposition.

In an example embodiment, BaTiO₃ nanocrystal synthesis may be conducted using a one-pot solvothermal approach based on the reaction between a barium titanium metalorganic source and an alcohol at a relatively low temperature of 180-230° C. and a self-generated pressure of 20-30 atm depending on the alcohols used and the reaction temperature. A BaTiO₃ bimetallorganic precursor may include barium titanium ethylhexano-isoproxide, a commercially available product, or can be prepared by dissolving metal Ba or metal oxide BaO with alcohol (ethanol, isopropanol, benzyl alcohol, oleyl alcohol or their mixture) following by mixing with equimolar quantity of titanium isopropoxides. Simple alcohol such as ethanol or isopropanol or their mixture may be used as a solvent. Addition of controlled amount of water (0-20 wt %) or alkaline (e.g. KOH) to the system can tune the rate of alcoholysis process, thus offering further controls over crystal size and dispersion in polar solvents.

The process enables the production of uniform and highly crystallized BaTiO₃ nanocrystals with high yields (>90%) and tunable sizes ranging from about 4 to 100 nm, depending on the barium titanium source, type of alcohol and the amount of water and alkaline in the system. The process can be scaled up to kilograms of production using a commercially available pressure reactor. The surface of intrinsically aggregate-free BaTiO₃ nanocrystals can be coated and functionalized with a variety of surface capping agents (e.g. oleic acid), surfactants, polymers or phospholipids, using a post-treatment process. Such BaTiO₃ nanocrystals retain good solubility and stability in either non-polar solvents (such as hexane, toluene) or polar solvents (such as ethanol, methanol).

The stable and homogeneous nanocrystal suspension enables easy incorporation of BaTiO₃ nanocrystals to other media (e.g. polymers, liquid crystals), which is favorable for either pure BaTiO₃ nanocrystal thin film processing or BaTiO₃-based nanocomposite thin film processing on both solid substrates (e.g. Si wafer) or flexible substrates (e.g. plastic) using a variety of available methods including coating (spin-coating, dip-coating, and cast-coating), printing or spraying techniques. Pure BaTiO₃ nanocrystal thin films are stable up to 500° C. with no sign of crystal merging and growth. BaTiO₃ nanocrystal thin films with a variable thickness ranging from 20 nm to 1 μm may be used in various applications. For example, BaTiO₃ nanocrystal thin films may be used as a ferroelectric component, a high dielectric constant, or high dielectric strength component of devices such as in capacitors, ultracapacitors, field-effect transistors, displays and other electronic devices.

The application of the various embodiments of nanoparticle based films may be implemented in various memory devices such as, but not limited to, non-volatile memory, volatile memory, FRAM, electrically erasable programmable read-only memory (EEPROM), and flash memory. Applications may include implementation in display devices, magnetic devices, giant magnetoresistance (GMR) devices including magnetoresistive random access memory (MRAM), magnetooptical devices, magnetooptical switching devices, electro-optic switching devices, waveguides, sensors, superconductors, membranes, and transparent conducting films. Applications may include devices, whose operation is based on properties of a material, for which an input such as electrical or magnetic fields, stress, heat or light yields an output such as charge, current, magnetization, strain, temperature or light as a consequence of a material property intrinsic to the structure of the material. Such properties include permittivity, permeability, elastic constant, specific heat, refractive index, piezoelectricity, the electro-calorific or magneto-calorific effects, the electro-optic or magneto-optic effect, the photoelastic effect, the pyroelectric effect, the photovoltaic effect, the piezomagnetic effect, thermal expansion, photostriction, superconductivity, the Faraday or Kerr effect. Applications may include implementations in catalysis or in a biological application. Various embodiments of nanoparticles may be implemented for an application based on a material device, for a material device based on a property of the material, for a material device based on a property of the material related to the structure of the material, for applications related to processing of a material to provide a desired structure of the material in a device, and for applications to process a material based on the ability to prepare the material from nanosized particles.

The family of complex oxide pervoskites (including BaTiO₃, Pb(Zr,Ti)O₃ and (Ba,Sr)TiO₃) possessing the ferroelectric property may be used in transducers and actuators (piezoelectric effect), high-K dielectric capacitors, and memory applications (microelectronics) that may rely on the hysteresis between two stable states of polarization. The investigation of micron to nanoscale ferroelectric materials (thin films and particles) has prompted a desire for a deeper understanding of how size effects polarization and ferroelectric order and the hypothesized importance of size effects in bulk ferroelectric systems. Contrasting views of the effect of sample size on ferroelectricity can be considered from experimental and theoretical points of view. First principles density-functional theory (DFT) provides a microscopic understanding of ferroelectrics. Theory can be used to calculate the relative stability of competing phases. A new understanding of the theory of bulk polarization along with new levels of theory may provide models for further materials based research.

Ferroelectricity, the existence of a remnant polarization, is a collective phenomenon influenced by surface and size effects. It is assumed that the macroscopic polarization, P, in the presence of applied electric field, E, is proportional to the displacement x of a set of ions from their position midway along some double well potential. The displacement gives rise to dipoles within the material, which can align to form domains with (P+, P−) or at some angle to the field. In Landau-Devonshire theory for a ferroelectric, the Helmholz free energy, F, can be expanded in a power series in the macroscopic order parameter for the polarization, P(T):

F(P,T,E)=−EP+AP²+BP⁴+CP⁶

where A=A₀(T−T_{c}). T_{C}, called the Curie temperature, can mark the transition between order and disorder in a ferroelectric material, corresponding to the transition between the ferroelectric and paraelectric phase respectively. When one introduces scaling dimensions, for example scaling to a thin film, one can analyze the effect of P(z) as a function of depth z into the ferroelectric and away from the electrode on the ferroelectric. Typically the effect is to lower T_{C} from that of the bulk by an amount roughly proportional to 1/d, where d is the film thickness.

The precise nature of critical size dependent suppression of T_{C} in a ferroelectric can be investigated in thin films prepared by deposition techniques and molecular beam epitaxy. Decreasing the thickness of thin films of perovskite ABO₃ compounds or decreasing the particle size in the nanoscale regime suppresses ferroelectricity to a critical size at which the property is eradicated.

Synthesis of uniformly sized and well isolated nanocrystals is helpful for addressing important fundamental issues such as of the size effect on their ferroelectric properties. Nanocrystals are isolated three dimensional nanometer scale units of materials, typically with symmetrical spherical or geometrical morphology and optimally, a well-formed crystalline core. The concepts of surface capping and solution stabilization have been developed to allow suspensions of nanoparticles to exist as solutions in a variety of aqueous and non-aqueous (organic solvent) media. The field can be aided greatly by the improved understanding of size-dependent scaling laws, which have emerged from fundamental studies in chemical physics and condensed matter physics. A wide variety of approaches for the preparation of BaTiO₃ nanocrystals, nanowires and nanotubes can be used, including pyrolysis of organometallic precursors, hydrothermal/solvothermal synthesis, coprecipitation and sol-gel processing. The solution-phase decomposition of bimetallic alkoxide precursors in the presence of coordinating ligands can yield well-isolated and single-crystalline BaTiO₃ nanocrystals and nanowires.

A synthetic strategy for preparing nano-structured complex oxides that retain ferroelectricity may be applicable to multiple roles in nanoelectronics. A sol-gel processing of bimetallic alkoxide precursor in presence of coordinating ligands such as oleic acid can yield uniform and well-isolated BaTiO₃ nanocrystals. Furthermore, monodispersed transition oxide nanocrystals can be synthesized by direct thermal decomposition of metal acetate in the presence of oleic acid at high temperature. A coordinating agent (e.g. oleic acid) plays a role in controlling the nucleation and crystallization of metal oxides by modifying the surface energy. A versatile synthetic procedure can involve the nucleation-controlled thermal decomposition of barium titanium molecular precursor in presence of oleic acid followed by further crystallization at higher temperature. With the control over the nucleation process and with the crystal surface capped with oleic acid, the resulting barium titanium oxide nanoparticles can be re-dispersed in hexane. Uniform BaTiO₃ nanocrystals may be produced in the form of isolated nanocrystals, continuous and micropatterned thin films by spin-coating or soft lithography (microprinting or micromolding). A high temperature hexagonal BaTiO₃ phase (which exists at 1460° C.) may be present at room temperature, probably due to cubic/tetragonal symmetries on nanometer scale. This versatile method may enhance the fundamental understanding of size-dependent evolution of ferroelectricity on individual nanocrystals and nanocrystal thin films and provided enhanced flexibility in integrating ferroelectric BaTiO₃ and other types of nanocrystals to electronic nanodevices.

In various embodiments, the film can be paraelectric. For some applications such as capacitors, the ferroelectricity is not always necessary for materials as long as they have high k dielectric constant. Sometimes, the ferroelectricity is undesirable, for example, high k gate dielectrics for transistor application. Accordingly, the film can be paraelectric but not ferroelectric.

Nano-structured thin films of barium titanate (BaTiO₃) can be built from uniform nanoparticles. The nanoparticles can be prepared by a chemical processing, based on thermal decomposition of a bimetallic barium titanium molecular precursor in the presence of oleic acid (a capping group), followed by high-temperature crystallization of this nucleation-controlled intermediate. Such a method offers a versatile way of preparing uniform and monodisperse BaTiO₃ nanocrystals, which can be used as a basis for micropatterned or continuous BaTiO₃ nanocrystal thin films. BaTiO₃ nanocrystals can crystallize with evidence of tetragonality. Well-isolated BaTiO₃ nanocrystals, smaller than 10 nm, can be prepared with control over aggregation and crystal densities on various substrates such as Si, Si/SiO₂, Si₃N₄/Si, Pt-coated Si substrates and other substrates. BaTiO₃ nanocrystal thin films may be formed with a uniform nanocrystalline grain texture. Electric field dependent polarization measurements show spontaneous polarization and hysteresis, indicating ferroelectric behavior for the BaTiO₃ nanocrystalline films with grain sizes in the range 10-30 nm. Dielectric measurements of the films show dielectric constants in the range 85-90 over the 1 KHz to 100 KHz, with low loss.

In an example embodiment for fabricating barium titanium oxide nanocrystals, preparation of a bimetallic barium titanium molecular precursor may include preparing barium titanium glycolate, BTG, (BaTi(C₂H₄O₂)₃4C₂H₆O₂H₂O). In a procedure for BaTiO₃ nanocrystal synthesis, a single bimetallic molecular precursor may be used to ensure a correct stoichiometry of the product. BTG may be first prepared in dry box by mixing BaO, ethylene glycol, 2-propanol and Ti(OPr)₄. The resulting white powder can be filtered, washed, dried at 60° C. and maintained in dry box because of its hygroscopic property. The BTG can be thermally decomposed in presence of oleic acid

Thermogravimetric (TG) analysis of BTG (BaTi(C₂H₄O₂)₃4C₂H₆O₂H₂O) is known to show three weight loss rate maxima at 67° C., 120° C., and 360° C., which correspond to the loss of single water molecule, four physically bonded ethylene glycol molecules, and three chemically bonded ethylene glycol molecules in a single BTG molecule, respectively. Based on the TG results, a thermal decomposition temperature of 360° C. can be used, at which three chemically bonded glycoalite ligands can be converted to oxide ligands in which barium titanium oxide starts to form. Oleic acid (OA) can be used as a coordinating agent to mediate the nucleation process. High boiling point solvent trioctylamine (TOA) (bp: 365-367° C.) can be used as a solvent to achieve such a high temperature.

In a process example, BTG powder (500 mg) can be first mixed with OA (1 ml) and TOA (12 ml) in a four-neck vessel. The BTG can be heating at 100° C. in N₂ until the BTG totally dissolves and a light yellowish clear solution is formed. The clear solution can be heated gradually in vacuum at 120° C., 200° C. and 250° C. to remove released ethylene glycol. Finally, the solution can be heated at 350-360° C. in a N₂ atmosphere for 2 h in the presence of oleic acid and trioctylamine until the solution turns to deep brown color. The barium titanium oxide nanoparticles can be separated by adding ethanol followed by centrifugation and washed by repeated precipitation/re-dispersion with ethanol. The resulting yellowish precipitate with OA coating can be re-dispersed in hexane with original concentration of ˜20 mg/ml and a clear orange solution can be obtained for further applications. X-ray diffraction measurement may show that the resulting yellowish precipitate may have an amorphous phase and not crystallized under the previous thermal treatment. In such a case, further high temperature treatment (600° C.) can be used to bring the nanoparticles into full crystallization.

BaTiO₃ individual nanocrystals and thin films can be formed on different substrates. For example, four different substrate include n-Si wafers with a native oxide layer, p-Si wafers with a SiO₂ layer (400 nm), p-Si wafers with a Si₃N₄ layer, and Pt(100 nm)/SiO₂(500 nm)/Si wafers. For preparation of micropatterned thin films, a clear hexane solution (barium titanium oxide nanoparticles diluted by 1:4) can be micro-printed or micro-molded on the substrates using a polydimethylsiloxane elastomer (PDMS) stamp. For preparation of individual BaTiO₃ nanocrystals, a dilute hexane solution can be spin-coated on substrates using a high spin rate (4000 rpm). To reduce the density of the BaTiO₃ nanocrystals and to improve the monodispersablity, the original barium titanium oxide nanoparticle solution can be diluted with hexane or with triblock copolymer Pluronic P123 (EO70PO20EO70) (1:2-1:16 by volume). For preparation of BaTiO₃ thin films, the original BaTi oxide solution can be spin-coated on substrates with a low spin-rate (1000-2000 rpm). As-prepared thin films can be subjected to 600° C. calcination in static air to achieve full crystallization. For preparation of triple or four-fold coatings, each coating can be dried at 300° C. before putting down next coating, where the multi-coated thin films can be eventually treated at 600° C. for 1 hour until fully crystallized.

Thin films of BaTiO₃ individual nanocrystals can be contacted with metal electrodes or electrodes of other conductive materials to form electrical structures in devices and to form test samples of the thin films. For example, a top platinum (Pt) electrode can be deposited by a vapor deposition system, such as a VEECO vapor deposition system with a four-position electron gun. The thin film samples may be maintained at room temperature while the Pt metal is heated by an electron beam to its melting temperature. The Pt vapor can be deposited through an aperture mask onto the thin film samples for 5 to 10 minutes in order to produce electrodes around 800 to 1000 Angstroms thick. The deposition can be carried out under vacuum down to 10⁻⁶ Torr. Other metals or conductive materials may be applied to thin films of BaTiO₃ individual nanocrystals to form conductive contact to the thin films using a variety of techniques, such as but not limited to, chemical vapor deposition, thermal evaporation, sputtering, atomic layer deposition, and combinations of various deposition techniques.

BaTiO₃ nanocrystals can be characterized with Scintag X2 X-ray Diffractometer using Cu Kα radiation (λ=1.54056 Å) and transmission electron microscopy (TEM, Philips EM 430). The BaTiO₃ nanocrystal thin films can be characterized also with scanning electron microscopy (SEM, Hitachi 4700 Hitachi 4700 Field Emission SEM) and atomic force microscopy (AFM, Nanoscope IIIa, Digital Instruments). For Raman scattering measurement, BaTiO₃ nanocrystals can be deposited onto a Si substrate and excited with the 488 nm line of an argon-ion laser focused to 2 μm. Unpolarized Raman scattered light can be collected in backscattering from ˜200 to 1200 cm⁻¹, with resolution better than 1 cm⁻¹. For electrical measurement of multi-coated thin films, top Pt electrodes, 0.1 mm in diameter and 50 nm in thickness can be evaporated on top of the films through a shadow mask by electron beam evaporation. Electric field-polarization hysteresis can be measured, for example, using a Radiant Precision Workstation. Frequency dependence of the capacitance and dielectric loss can be measured by a HP4194A Impedance Gain/Phase Analyzer in the low frequency region of 1 KHz to 100 KHz. Dielectric constant εᵣ can be calculated by the formula C=ε₀εᵣA/d, where C is the capacitance, A is the area of the electrode, d is the film thickness, and ε₀ is the dielectric constant of air.

Although the TG analysis of BTG has shown the highest peak temperature to convert chemically bonded glycoalite ligands to oxide ligands is 360° C., the thermal treatment at 360° C. in the presence of oleic acid and TOA may not fully decompose the BTG to crystallize BaTiO₃. Powder X-ray diffraction (XRD) results may show that the barium titanium oxide nanoparticles are still in an amorphous phase after a first-step thermal decomposition. Thus, a second-step thermal treatment at higher temperature (e.g. 600° C.) may be performed to fully crystallize the BaTiO₃. The XRD patterns show that after 600° C. treatment, all diffraction peaks can be assigned to BaTiO₃ phase without any indication of other crystalline by-products such as barium carbonate or titanium dioxide. The diffraction peaks are broad, indicating the formation of nanocrystals. The average crystal size can also be calculated as 13.4 nm in average from Debye-Scherrer equation by taking account of the peak broadening at (111) diffraction line (no peak splitting due to symmetry change). In addition, the diffraction pattern in the 2 theta=40-50° region is usually characteristic of the presence of either cubic or tetragonal BaTiO₃ structure. In this case, no splitting of cubic (200) into tetragonal (200) and (002) reflections at about 45° can be observed. It is also possible that the reflections are too broad to distinguish between the cubic and tetragonal modification of perovskite BaTiO₃. The lattice parameter can be estimated to be 4.014 Å from the diffraction peaks at (100) and (200) and the ratio of c/a can be estimated to be 1.0032 from the average ratio of diffraction peak (111) vs diffraction peaks at (100), (110) and (200), respectively. Compared with a pure tetragonal phase (c/a=1.01), it shows that the BaTiO₃ nanocrystals are crystallized with some degree of tetragonality although there is no obvious splitting of (200) peak due to the size effect. The XRD pattern also shows that beside the formation of perovskite BaTiO₃ with cubic/tetragonal symmetries, there are some very small and broad peaks, which can be assigned to BaTiO₃ in hexagonal symmetry. The hexagonal BaTiO₃ phase usually exists at high temperature (1460° C.). High-resolution transmission electron microscopy can be used to study the BaTiO₃ nanocrystals prepared from the decomposition of metal-organic precursor. A high-temperature hexagonal phase can be attributed to a locally stacked hexagonal sequence in cubic-tetragonal crystallites, as a result of nano-sized twins in the cubic-tetragonal matrix. Similarly, the presence of hexagonal symmetry here can be attributed to the coexistence of cubic/tetragonal phases at nanometer scale. The tetragonality in BaTiO₃ nanocrystals together with the presence of the hexagonal symmetry can also be confirmed with a Raman scattering measurement.

Compared with the long-range ordering structure revealed by XRD measurement, Raman scattering spectra are more sensitive to short-range ordering structure. The Raman spectra show a small shoulder peak at around 307 cm⁻¹ and a very broad peak at ˜710 cm⁻¹ at room temperature, which are characteristic of structural tetragonality (4 mm symmetry). Moreover, temperature-dependent Raman spectra show these two peaks still persist when temperature is raised above a Curie temperature (ferroelectric-paraelectric transition temperature recorded around 125° C. for bulk BaTiO₃), indicating no distinct phase transition for BaTiO₃ on such a small nanometer range. After 850° C. annealing for 1 h, the two Raman peaks become more distinct because of increased tetragonal symmetry although there is still no obvious splitting of the XRD diffraction peak at (200). Besides the two characteristic peaks for the tetragonal phase, an additional Raman peak near 630 cm⁻¹ can also be observed, and it still persists with reduced intensity after the 850° C. annealing. This peak around 630 cm⁻¹ could be attributed to the following possibilities: (1) Ti—O stretch of titanium-based oxide phase; (2) carbonate species, (3) strain in grain-boundary regions, and (4) hexagonal BaTiO₃ phase. Since no carbonate and titanium species are found in the XRD pattern and no strong Raman peak around 400 cm⁻¹ found for anatase-like phase, the first two possibilities may be ruled out. The carbonate species usually disappears after 750° C. annealing, however, the peak near 630 cm⁻¹ still persists with reduced intensity after the annealing at 850° C. Since the above XRD results suggested a nanostructure with cubic/tetragonal symmetry along with the presence of hexagonal symmetry, it appears reasonable to assign the peak 630 cm⁻¹ to the presence of hexagonal phase due to the proposed nanotwin structures, which may cause the strain in the grain-boundary as well. This is consistent with other reports, especially when BaTiO₃ was prepared from the decomposition of metal-organic molecular precursors. The distinct peak at 630 cm⁻¹ can be attributed to a high-temperature hexagonal phase coexisting with the cubic-tetragonal phase at room temperature due to size effects.

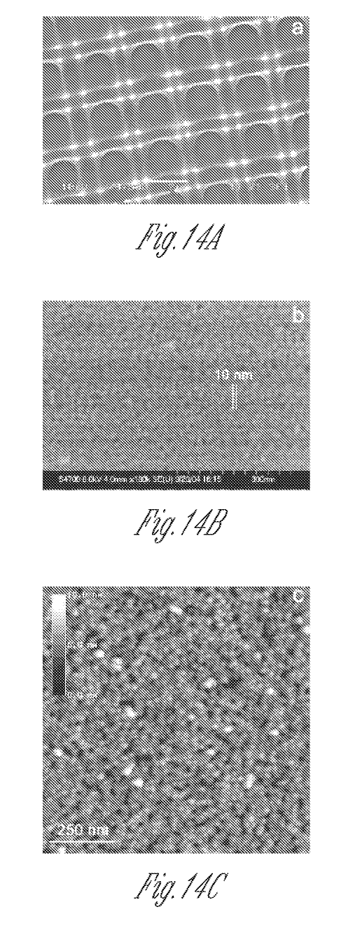

In various embodiments, BaTiO₃ thin films may be prepared from nanocrystals. Soft-lithography techniques including micromolding in capillaries (MIMIC) and microcontact printing using PDMS stamps can be successfully applied to pattern a variety of materials (e.g. organic molecules, polymers, proteins, nanoparticles, colloids and metals). As an example, BaTiO₃ nanocrystals are micro-patterned using micromolding of a barium titanium oxide nanoparticle solution followed by 600° C. crystallization. FIG. 14a shows a BaTiO₃ thin film with a micro-pattern, which further shows that the pattern is made of BaTiO₃ nanocrystals. A variety of patterns can be obtained by choosing PDMS molds with appropriate micropatterns. In addition, continuous BaTiO₃ thin films can be prepared by spin-coating of original concentration of the barium titanium oxide nanoparticle solution with relative slow spin rates (1200-2000 rpm) followed by 600° C. crystallization. Uniform thin films can be obtained with their colors depending on the film thickness. In FIG. 14b, the high-resolution SEM image shows the thin film is composed of uniform BaTiO₃ nanocrystals whose grain size is about 10 nm and visible inter-crystal voids as well, which probably arise form the loss of organic species during the thermal treatment for crystallization. A typical film may contain 10% to 15% porosity. There are a few sporadic larger nanocrystals (˜25 nm, less than 1%) among the small nanocrystals. The crystal size is in good agreement with TEM observation on the powder samples. The tapping-mode AFM image in FIG. 14c also confirms the uniformity of the BaTiO₃ nanocrystals in the thin film. The uniformity of the nanocrystals also depends on the substrate used. Experiments show that BaTiO₃ nanocrystals are uniform when dispersed on Si substrates, while they become less uniform on Pt-coated Si substrates.

To be perform electrical measurements on BaTiO₃ thin films, a 100 nm thick Pt coated Si wafer with 500 nm thick SiO₂ layer [Pt(100 nm)/SiO₂ (500 nm)/Si] can be used as a substrate, where the Pt coating can be used as a bottom electrode and e-beam evaporated Pt dots on top of BaTiO₃ thin films through a shadow mask can be used as top electrodes. As discussed above, since the BaTiO₃ single coating may be full of inter-crystal voids, triple or four coatings may be applied on the Pt(100 nm)/SiO₂ (500 nm)/Si substrates to make the film thicker and denser, so that it can reduce possible electrical shortcut created on the evaporation of top Pt electrodes. Smaller electrode area may be used. However, the multi-coatings usually afford less uniform nanocrystals and larger crystal size distribution (for instance, the average grain size is around 10-15 nm for triple coatings, and 20-30 nm for four coatings.) Although the less uniformity over micron scale, the low SEM magnification and the optical microscopy show that the BaTiO₃ film is still quite uniform over a macroscopic range except for very few sporadic structural pinholes on the film surface.

Representative polarization-electric field hysteresis curves of the BaTiO₃ thin films (triple coatings and four coatings) show obvious hysteresis loops when sweeping the electric field across the films. For the triple BaTiO₃ coatings (˜70 nm in thickness, 10-15 nm in grain size), the film is stable up to an electric field of 700 kV/cm (couples of cycles) and starts to break down when driving the electric field higher than 800 kV/cm. The leaky behavior is believed to originate from the small inter-crystal voids inside the film. The BaTiO₃ thin film becomes thicker and denser after four coatings (˜108 nm in thickness, 20-30 nm in grain size), and it shows constant ferroelectric hysteresis loop and can be stable up to an electric field of 900 kV/cm without any sign of breakdown. The remnant polarization value Pᵣ and the coercive field value E_{c} of the BaTiO₃ film with four coatings are around 1 μC/cm² and 90 kV/cm, respectively, compared to the reported single crystal value (Pᵣ=24 μC/cm² and E_{c}=1.5 kV/cm) and the reported ceramics value (Pᵣ=8 μC/cm² and E_{c}=3 kV/cm). The polarization of the BaTiO₃ film with four coatings did not reach saturation due to the increased chance of leakage at higher electrical fields. However, the spontaneous polarization, Pₛ, of the BaTiO₃ film with four coatings can be estimated by extrapolating the tip of the hysteresis to be around 8 μC/cm², while the reported Pₛ value for a good single crystal is 26 μC/cm² and the reported Pₛ value for ceramics is around 13 μC/cm². A significant decrease of the Pₛ and Pᵣ in the case of films that exhibit nanocrystalline texture is anticipated, in accordance with previous observations and theoretical predictions when the grain size is reduced to nanoscale. Both thin films show similar electric field dependent polarization behaviors, and the observed hysteresis loops also indicate that the BaTiO₃ nanocrystals with varying size between 10-30 nm are all ferroelectric, as a result of the tetragonality of the nanocrystals. In addition, the shape of the hysteresis loops of the BaTiO₃ films does not show much change after 850° C. treatment for 1 h, indicating that ferroelectricity of the tetragonal BaTiO₃ nanocrystals evolves at temperature as low as 600° C.

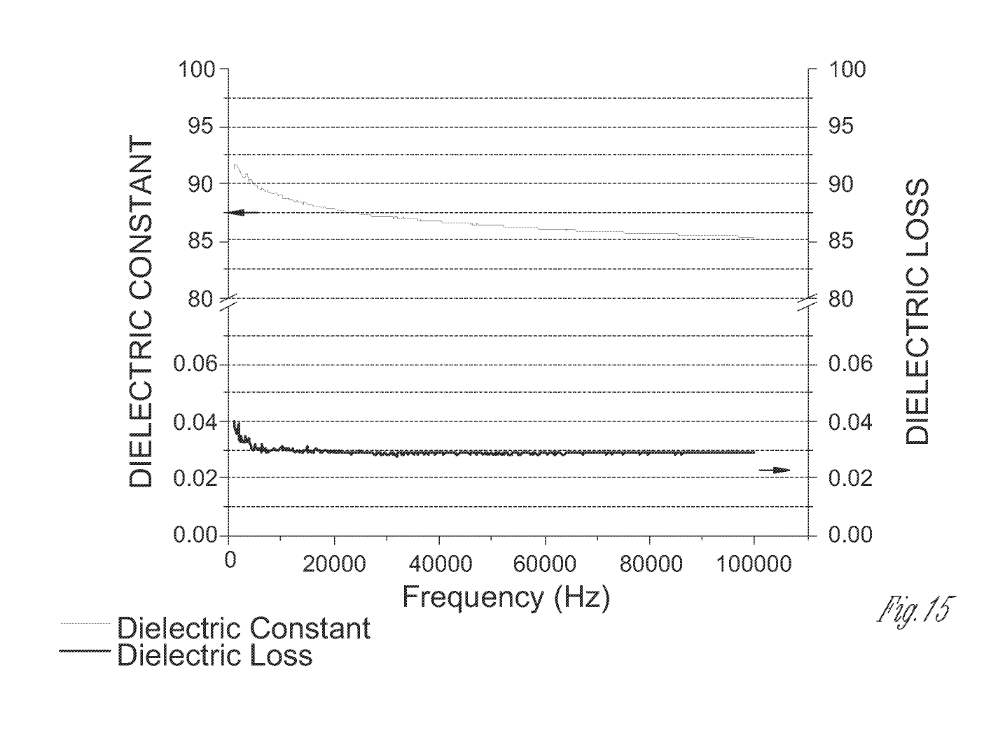

FIG. 15 shows dielectric constant and dielectric loss over the frequency range of 1 KHz to 100 KHz for the BaTiO₃ thin film (four coatings). In FIG. 15, the typical dielectric constant of the thin film is around 85 to 90 with the dielectric loss around 0.03 or below in the whole frequency range. This value is significantly lower than the reported bulk BaTiO₃ ceramics value (1500 at 1 kHz). However it is relatively high when compared to other reported values, taking into consideration (a) ˜15% porosity in the film and (b) the nanoscale grain size. Moreover, when the frequency is below 100 KHz, the dielectric constant increases very slightly while the dielectric loss remains very low (0.03 to 0.04) in the whole frequency region. This suggests that very few conducting carriers such as ionic space charge carriers exist in these BaTiO₃ thin films. In single crystal barium titanate, the dielectric constant is highly anisotropic, due to the fact that the displaced atoms are tightly bound by the ferroelectric displacement along the polar axis (c-axis), while they are relatively free to vibrate in the perpendicular direction. This is not the case for nanocrystalline thin films in which the nanocrystallites are randomly oriented and thus an averaged value of the dielectric constant over the crystallographic axes is predicted.

In various embodiments, a nucleation-mediated barium titanium oxide intermediate method involving the thermal decomposition of barium titanium organometallic precursors in presence of capping agent oleic acid affords uniform BaTiO₃ nanocrystals after 600° C. calcinations. This method offers a versatile means of preparing well-isolated, patterned or continuous thin films of BaTiO₃ nanocrystals on various substrates, and can be easily incorporated with current micro- and nanofabrication processes. The BaTiO₃ nanocrystals are crystallized with some degree of tetragonality, which is the source of the ferroelectricity found in such small size range. Electric field dependent polarization measurements show spontaneous polarization and hysteresis, indicating ferroelectric behavior for the BaTiO₃ nanocrystalline films with grain sizes in the range 10-30 nm. Dielectric measurements of the films show dielectric constants in the range 85-90 over the 1 KHz to 100 KHz, with low loss, which may provide enhanced performance in use in thin film capacitance applications or other electronic device applications.

Organic field effect transistors (OFETs) can be prepared using barium titanate thin films as high-K dielectric layers. For example, the inventors herein have fabricated pentacene OFETs with a spin-cast barium titanate (BaTiO₃) nanoparticle thin film (e.g., K˜40 measured in N₂) layered with a coating of parylene-C. Because the size (e.g., ˜8 nm) of BaTiO₃ crystals is much smaller than the domain size, the ferroelectric hysteresis seen in bulk BaTiO₃, which depends on domain wall movement, can be eliminated. In an example, the fabrication process can occur at a temperature that is below 60 degrees C. The linear region mobility of the OFET can be improved, such as by about one order of magnitude against a control arrangement. Without being bound by theory, it is believed that this can be due to the higher accumulated charge carrier density achievable because of the higher dielectric-constant gate dielectric.

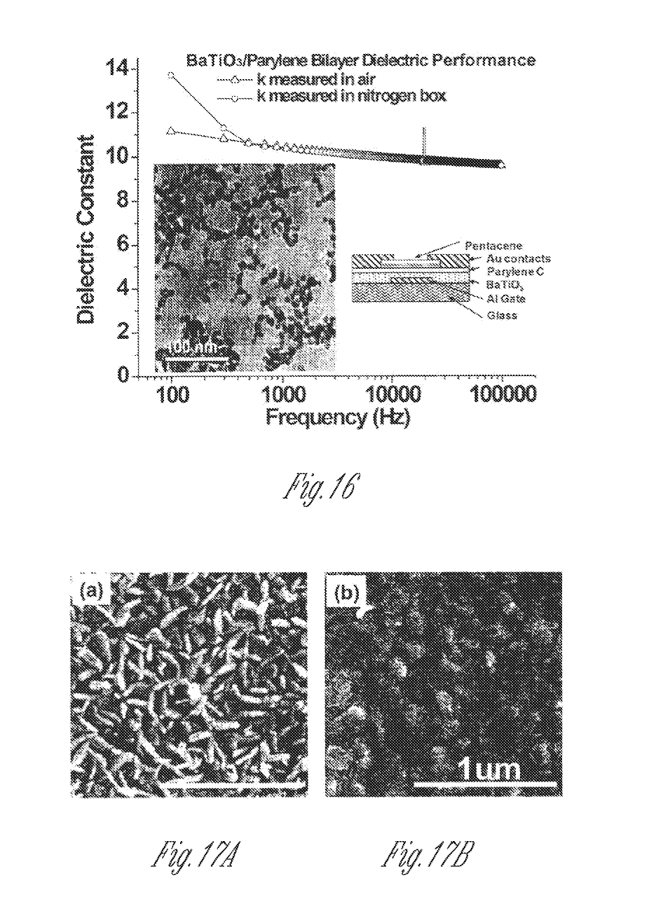

In an example, the BaTiO₃ nanoparticles were synthesized using a solvothermal process, which can produce uniform and aggregate-free BaTiO₃ nanocrystals (see, e.g., FIG. 16 TEM image) with tunable size. The OFETs can have a bottom-gate top-contact structure (see, e.g., FIG. 16 schematic cross section). In an example, an 8 nm diameter BaTiO₃ ethanol suspension (e.g., 20 mg/ml) was spin-coated, such as at 1000 rpm, on a detergent-cleaned glass substrate with patterned Al gate electrodes. The sample was baked at 60 degrees C. for two hours to remove the residual solvent. The thickness of the BaTiO₃ film was measured, such as by cross-sectional scanning electron microscopy (SEM). A 90 nm thick parylene-C layer was applied, such as using a CVD system.

Equivalent metal-insulator-metal (MIM) capacitor structures with the same BaTiO₃/parylene dielectric were simultaneously fabricated, and their dielectric properties were analyzed, such as by using an Agilent 4284A capacitance bridge in air and under dry N₂ (see, e.g., FIG. 16). The effective dielectric constant of the composite dielectric was 11 at 100 Hz (see, e.g., FIG. 16). In an example, 25 nm of purified pentacene (e.g., from Luminescence Technology) was deposited and patterned, such as using a shadow mask at the deposition rate of 0.1 A/s, with the substrate held at room temperature at a pressure less than 5×10⁻⁷ Ton. In an example, 45 nm thick Au source/drain electrodes were deposited, such as using shadow masking. A set of OFETs with similar structure but using a gate dielectric composed of 110 nm parylene-C was used as a control sample. The characteristics of the resulting OFETs were measured, such as by using an Agilent 4155C semiconductor parameter analyzer. More than 100 OFETs can be produced on each substrate; typical characteristics of devices are shown. The bare BaTiO₃ layer, without the parylene coating, can exhibit a higher dielectric constant (e.g., about 35 to 40, measured in N₂) than the composite film. The layer of parylene-C (e.g., K˜3.15), however, can promote favorable growth of the pentacene and can allow transistors to be formed.

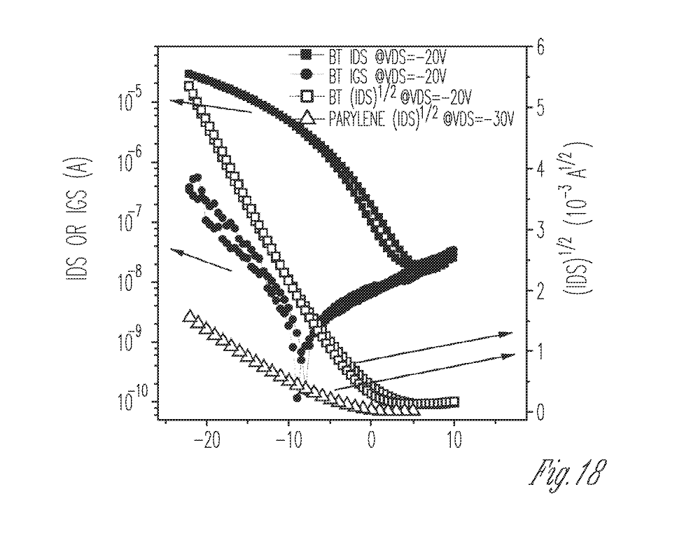

FIG. 17 illustrates a comparison of pentacene grown on (a) bare BaTiO₃ thin film and (b) parylene-C coated barium titanate thin film, imaged by SEM. The scale bars in both images are 1 μm. On bare BaTiO₃, the pentacene grains fail to form a continuous film and exhibit a large fraction of vertical grains. No measurable transistor behavior is observed in these samples. Pentacene forms a smooth layer on the parylene-C coated BaTiO₃. The parylene layer also suppresses some gate leakage, while still allowing for a reasonably high effective dielectric constant.

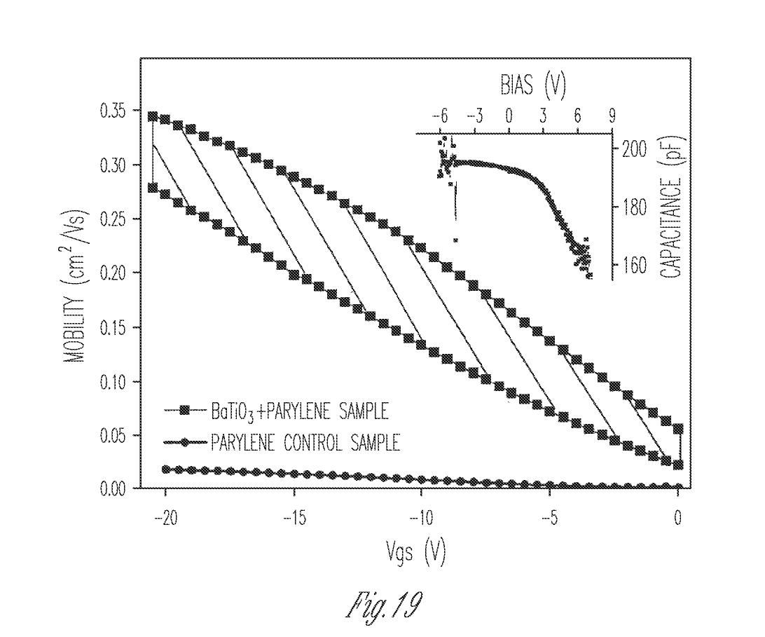

FIG. 18 illustrates a plot of OFET drain-source current, I_{Ds} (represented by solid squares), √I_{DS} (represented by open squares), and gate leakage I_{GS} (represented by open circles), each vs. the gate source voltage V_{GS} of the BaTiO₃/parylene OFETs in the saturation region. Also shown in FIG. 18 is the √I_{DS} (represented by open triangles) of parylene-only OFETs in the saturation region. In the example of FIG. 18, the dielectric may still low more gate leakage than may be desirable for some applications, but minimal polarization hysteresis is seen in the BaTiO₃/parylene OFETs, confirming that the polarization hysteresis seen in bulk material is quenched due to the small size of BaTiO₃ nanocrystals.

FIG. 19 illustrates an example of linear mobility in OFETs with 110 nm parylene only (represented as dots) as gate dielectric and the ones with BaTiO₃/parylene (represented as squares) as gate dielectric. In an example, the capacitance of 110 nm parylene-C is 25 nF/cm², and that of the composite dielectric is 31 nF/cm². Inset in FIG. 19 is a graph of QSCV of a BaTiO₃/parylene OFET.

In OFETs, the current-fitting equation I_{DS}=W/L Cᵢμ(V_{GS}−Vₜ)² is not ideal for device characterization in saturation, because of the strong mobility dependence on the gate field and the poor definition of the threshold voltage in disordered semiconductors. We have applied the method by K. Ryu, I. Kymissis, V. Bulovic, and C. G. Sodini, “Direct extraction of mobility in pentacene OFETs using C−V and I−V measurements,” IEEE Electron Device Letters, 26(10):716-718, (2005), to calculate the mobility in the linear region, which has a more direct physical meaning. In an OFET, the mobility can be extracted from the fundamental relationship:

$$μ = \frac{v}{E} ; v = \frac{{I}_{DS}}{WQ} ; E = \frac{{V}_{DS}}{L} \qquad ( 1 )$$

where ν is the velocity, E is the electrical field, W is the channel width (for our example, W=800 μm), L is the channel length (for our example, L=60 μm), and Q is the induced charge per channel area.

$$Q = {\int }_{+ ∞}^{{V}_{GS}} ( \frac{{C}_{ch}}{WL} ) d V ; {C}_{ch} = {C}_{OFET} - {C}_{dep} \qquad ( 2 )$$

where C_{ch} is the channel capacitance due to the charge induced in the pentacene semiconductor, C_{OFET} and C_{dep} are the capacitance of the OFET under different gate voltages and completely depleted OFET. Both the values can be obtained from independent quasi-static C−V (QSCV) measurements (e.g., shown as FIG. 19 inset).

Using the transfer characteristic in the linear region (not shown), the mobility (represented by squares) of BaTiO₃/parylene OFETs was calculated versus gate voltage (see, e.g., FIG. 19) according to Eq. (1) and Eq. (2). For comparison, the mobility (represented by dots) for OFETs with parylene-only as gate dielectric was also drawn. The mobility in both cases had a strong dependence on the gate voltage. At V_{GS}=−20V, the mobility for OFETs with the BaTiO₃/parylene-C gate dielectric was ˜0.35 cm²/(V·s), about one order of magnitude larger than that in OFETs with parylene-C only as gate dielectric, which was ˜0.03 cm²/(V·s). Assuming the mobility of the former can be extended to further negative gate bias in a linear scheme without any saturation, the value of mobility at V_{GS}=−40V is around 0.6˜0.7 cm²/(V·s). Although this method of calculating mobility usually gives a lower value than the traditional curve fitting method, it provides more accuracy.

The increased concentration of accumulated carriers and pentacene crystal quality are not solely responsible for enhancing the field effect mobility in the transistors. The BaTiO₃/parylene OFETs generated more charges, e.g., 0.61 μC/cm² at V_{GS}=−20V, while the control sample generated 0.51 μC/cm². The dependence of mobility on accumulated charge is comparable to the references. Other factors, such as the pentacene crystalline quality and the effect of the dielectric constant on charge injection, can also contribute to an effective mobility difference.

The use of a stacked gate dielectric film, such as by using solution-deposited 8 nm-diameter high-K BaTiO₃ nanocrystals together with parylene-C to fabricate pentacene-based OFETs, as has been disclosed herein, enhances the mobility of the semiconductor by increasing the concentration of accumulated carriers and suppresses the ferroelectric hysteresis of the BaTiO₃ by using particles smaller than the domain size. In certain examples, our structure can have one or several advantages, such as including, for example, a high-dielectric constant with relatively small hysteresis, low temperature processing (e.g., <60 degrees C.), applicability for large area fabrication, and applicability to low thermal budget flexible substrates. Further improvements can be obtained by decreasing the gate leakage and further increasing the hybrid stack capacitance.

It is to be understood that while the invention has been described in conjunction with the detailed description thereof, the foregoing description is intended to illustrate and not limit the scope of the invention, which is defined by the scope of the claims. Other aspects, advantages, and modifications are within the scope of the claims and will doubtless be apparent to persons of ordinary skill in the art.

### EXAMPLES

### Example 1

### Synthesis of BaTiO₃ Nanocrystals with Diameters of 6-10 Nm (Capped with Decanoic Acid) (Case I)

A bimetallic precursor solution is prepared by dissolving 1 mmol metallicbarium (99.99% ESPI) in 10 mL anhydrous benzyl alcohol C₆H₅CH₂OH (99.8% Aldrich) in a glovebox at 80° C. with stirring until a slightly yellow transparent solution is formed. Then, the transparent solution is cooled down to room temperature and an equimolar quantity of titanium (IV) isopropoxide Ti[OCH(CH₃)₂]₄ (99.999% Aldrich) is added and mixed at 30-50° C. until a white precipitate is formed. The white precipitate (amorphous) is used as the bimetallic precursor in solution without further treatment. The bimetallic precursor solution is then transferred with a syringe out of the glove box and injected immediately through a septum into a preheated (320° C.) solvent/ligand mixture. The composition of the solvent/ligand mixture includes oleylamine, (CH₃(CH₂)₇CH═CH(CH₂)₇CH₂NH₂, 45 mL, Aldrich), and decanoic acid, (CH₃(CH₂)₈COOH, 0.9 g, Aldrich) (case I, ON/DA). The solvent/ligand mixture had been preheated to 320° C. with stirring under dry nitrogen flow and at ambient pressure. Right after injection, the temperature drops to around 220° C. with the boiling of low boiling point benzyl alcohol, which is carried away gradually by the nitrogen flow. Within five minutes after injection, the mixture of the white precursor solution, hot oleylamine and carboxylic acid turns from cloudy (right after injection) into a transparent, slightly yellow solution. This is attributed to the high solubility of the precursor in the solvent/ligand mixture. Within 60 to 120 minutes, the benzyl alcohol is removed under the N₂ flow, and the temperature of the system achieves its final resting temperature of 320° C. This temperature is maintained for 24 hrs to prepare decanoic acid capped 6-10 nm BaTiO₃ nanocrystals;

### Example 2

### Synthesis of BaTiO₃ Nanocrystals with Diameters of 3-5 Nm (Capped with Oleic Acid) (Case II)

The bimetallic precursor solution is prepared by dissolving 1 mmol metallic barium (99.99% ESPI) in 10 mL anhydrous benzyl alcohol (C₆H₅CH₂OH, 99.8% Aldrich) in a glovebox at 80° C. with stirring until a slightly yellow transparent solution is formed. Then, the transparent solution is cooled down to room temperature and an equimolar quantity of titanium (IV) isopropoxide (Ti[OCH(CH₃)₂]₄, 99.999% Aldrich) is added and mixed at 30-50° C. until a white precipitate is formed. The white precipitate (amorphous) is used as the bimetallic precursor in solution without further treatment. The bimetallic precursor solution is transferred through a syringe out of the glove box and injected immediately through a septum into a preheated (320° C.) solvent/ligand mixture. The composition of the solvent/ligand mixture included oleylamine (45 mL) and oleic acid, (CH₃(CH₂)₇CH═CH(CH₂)₇COOH, 1 mL, Aldrich) (case II, ON/OA). The solvent/ligand mixture had been preheated to 320° C. with stirring under dry nitrogen flow and at ambient pressure. Right after injection, the temperature drops to around 220° C. with the boiling of low boiling point benzyl alcohol, which is carried away gradually by the nitrogen flow. Within five minutes after injection, the mixture of the white precursor solution, hot oleylamine and carboxylic acid turns from cloudy (right after injection) into a transparent, slightly yellow solution. This is attributed to the high solubility of the precursor in the solvent/ligand mixture. Within 60 to 120 minutes, the benzyl alcohol is removed under the N₂ flow, and the temperature of the system reverts to its final resting temperature of 320° C. This temperature is maintained at 48 hrs to prepare oleic acid capped 3-5 nm BaTiO₃ nanocrystals.

### Example 3

### Synthesis BaTiO₃ Nanoparticles and Nanorods with Size of 10-20 Nm (Capped with Oleyl Alcohol) (Case III)

BaTiO₃ nanocrystals were also prepared by replacing BzOH by a mixture of 9 mL BzOH and 1 mL oleyl alcohol (OLOH) (CH₃(CH₂)₇CH═CH(CH₂)₇CH₂OH, Aldrich), (case III). No carboxylic acid is added as a ligand. In a typical synthesis, 2 mmol metallic barium is transferred from a glove box and added to alcohol (case III: 9 mL BzOH and 1 mL OLOH with stirring under dry N₂ flow. Then the temperature of this system is elevated to 80° C. and maintained at this temperature until barium is dissolved into the alcohol and a transparent solution is formed. Then the solution is cooled down to room temperature and equimolar titanium isopropoxide is injected through a septum for overnight mixing at room temperature. This solution is used as the bimetallic precursor solution without further treatment. Oleylamine (45 mL) is injected through a septum into this precursor solution with no further addition of carboxylic acid. After injection the temperature of the system is brought slowly (2-3 hrs) to 320° C. (in both case III and case IV) with stirring under N₂ and rest at this temperature for 24 hrs (case III) or 48 hrs (case IV).

### Example 4

### Synthesis of BaTiO3 Nanocrystals of about 2-3 Nanometers Diameters (Capped with Oleyl Alcohol and Oleylamine) (Case IV)

BaTiO₃ nanocrystals were also prepared by replacing BzOH with 10 mL OLOH (case IV). No carboxylic acid is added as a ligand. In a typical synthesis, 2 mmol metallic barium is transferred from a glove box and added to alcohol (10 mL OLOH) with stirring under dry N₂ flow. Then the temperature of this system is elevated to 180° C. and maintained at this temperature until barium is dissolved into the alcohol and a transparent solution is formed. Then the solution is cooled down to room temperature and equimolar titanium isopropoxide is injected through a septum for overnight mixing at room temperature. This solution is used as the bimetallic precursor solution without further treatment. Oleylamine (45 mL) is injected through a septum into this precursor solution with no further addition of carboxylic acid. After injection the temperature of the system is brought slowly (2-3 hrs) to 320° C. with stirring under N₂ and rest at this temperature for 48 hrs.

### Example 5

### Recovery of Capped Nanocrystals

The BaTiO₃ nanocrystals are collected by adding polar solvents, ethyl alcohol (case I and III) or acetone (case II and IV) for particle precipitation and subsequent centrifugation. Extra solvent and surfactant can be removed and BaTiO₃ nanocrystals are collected by the following way: acetone or ethanol is added to the reaction solution in the volume ration around 3:1 or greater. With shaking or sonicating, the transparent solution becomes cloudy. This cloudy solution is sonicated for one minute to ensure thorough mixture of polar solvent and solution, then centrifuged at high speed (13.4 Krpm) to collect the precipitate. After high speed centrifugation, the precipitate is collected and dispersed in hexane or toluene by shaking or sonication for one to two minutes. This procedure is generally referred as “washing” nanoparticles to remove extra solvent or surface ligands. Then polar solvent can be added into this hexane or toluene solution to “wash” the nanoparticles for the second time if needed. Because the extra solvent and surface ligands, the BaTiO₃ nanoparticles capped with oleic acid or oleyl alcohol need to be “washed” for at least twice or more. As the nanocrystals are “washed” for more and more times they turn whiter and whiter and the surface ligands can be eventually stripped off, which can affect solubility in non-polar solvents. After washing, the nanocrystal precipitates can be dispersed in to nonpolar solvents such as hexanes or toluene to form a transparent slightly yellow solution with the help of sonication for three to five minutes. The nanocrystals are easily re-dispersed in nonpolar solvents such as hexane or toluene.

### Example 6

### Characterization of Nanocrystals

Transmission Electron Microscopy (TEM) and Powder X-ray Diffraction (XRD) characterizations were carried out on as synthesized nanocrystals. Results are shown in FIGS. 1-3 Low magnification conventional TEM is performed on a JEOL 100CX microscope and High Resolution TEM (HRTEM) is performed on a JEOL JEM 3000F microscope. XRD is performed on an Inel Multipurpose diffractometer by drop coating particle solution on a glass substrate. IR data (FIG. 4) and NMR data (FIG. 5) of the bimetallic precursor solution are also shown.

### Example 7

A reaction between a barium titanium metalorganic source and an alcohol (such as ethanol, isopropanol, the alcohol mixture, or alcohol with a controlled amount of water (e.g. 95% ethanol (“azeotrope ethanol”)) is carried out at a temperature of 80-230° C. The reaction takes place in an autoclave where self-generating pressure (20-30 atm) from alcohol vapor at elevated temperatures can enhance the reactivity at the low temperature. The BaTi bimetallorganic source is commercially available or is prepared by dissolving Ba metal or barium oxide (BaO) in ethanol or isopropanol, or the alcohol mixture followed by mixing with an equimolar quantity of titanium isopropoxide (molar ratio of Ba:Ti=1). The alcohol is a good solvent that can dissolve the barium titanate precursor. The alcohol is also a reactant involved in the alcoholysis process or in the reaction with an organic moiety of the metalorganic source, which is believed to initiate formation of Ba—O—Ti bonds, a crucial step for the nucleation of BaTiO₃ nanocrystals. The alcohol can also act as a surface modifier to stabilize the nanocrystals by forming ether end groups on the nanocrystal surface. It is also found that trace amount of water such as in 95% ethanol can tune the rate of alcoholysis process, thus offering further controls over the particle size and morphology. Water also can modify the crystal surface with more hydroxyl groups, providing for better solubility of BaTiO₃ nanocrystals in polar solvents such as ethanol, with no aid from other additives or surfactants. The BaTiO₃ nanocrystals are easily dispersed in ethanol to afford a substantially homogeneous suspension that can be stable up to weeks or longer without precipitation. Only a small number of nanoparticles may precipitate out in a month, but they can be easily re-dispersed in ethanol using sonication. Other nonaqueous processes that take place in high-boiling-point solvents usually produce BaTiO₃ nanocrystals with surface ligands and some contamination from the solvents. For most of thin film applications, the organic coating must be removed by oxidation in order to retain the intrinsic properties of the nanocrystals. However, the organic residue after the oxidation (mainly carbon-rich species) can become a major contributor to electrical leakage. In addition, when the organic species is removed, some voids may be left, resulting in high porosity, that results in a low dielectric constant. For example, the value of the dielectric constant k can be about 90 for a calcined thin film vs. about 750 for a pure BaTiO₃ nanocrystalline thin film. Thin films with voids also have poorer mechanical strength. On the contrary, the simple reaction system containing only a BaTi metalorganic source and a general alcohol can produce high purity, high crystalline BaTiO₃ nanocrystals. The product can be easily collected and cleaned with little or organic residue. The resulting nanocrystals can be easily dispersed in ethanol with no additives or surfactants. The stable dispersion can provide highly pure BaTiO₃ nanocrystalline thin films with minimal contamination from organic ligands or solvents.

### Example 8

### Synthesis of Uncapped, Uncoated BaTiO₃ Nanocrystals

The synthesis of barium titanate nanocrystals is based on the solvothermal process of a BaTi metalorganic source (barium titanate precursor) in an alcohol solvent. The first step of the synthesis was performed in a glove box because of the moisture-sensitive nature of the sources. BaTi ethylhexano-isoproxide, a commercially available product, is used as a precursor. The precursor was also prepared by dissolving metal Ba or metal oxide BaO in anhydrous ethanol or isopropanol or the alcohol mixture followed by mixing with equimolar quantity of titanium isopropoxide (molar ratio of Ba/Ti=1) and certain amount of alcohol solvent (ethanol, isopropanol, or alcohol with controlled amount of water). The homogeneous solution (or suspension) was transferred to an autoclave and heated in oven at a temperature of between 180 and 220° C. with self-generating pressure (20-30 atm) for a desired period of time (1-4 days).

### Sample A:

0.325 g metal barium was dissolved in 20 ml anhydrous ethanol or isopropanol to form barium alkoxide clear solution with concurrent release of hydrogen. 0.7 ml titanium isopropoxide was added dropwise to the solution under stirring. The clear solution was stirred for 5 minutes for the preparation of a BaTi metalorganic source. Then, 20 ml 95% EtOH solvent (pre-boiled to remove CO₂) was added to the solution. The clear mixture was stirred for 5 minutes and then transferred into a Teflon-lined stainless steel autoclave (Parr Instrument Company). The autoclave was taken out of the glove box and heated at 210° C. for 48 hrs.

### Sample B:

16 ml BaTi ethylhexano-isoproxide was dropwise added to 20 ml 95% EtOH under stirring. The opaque solution was stirred for 5 minutes and then transferred into the autoclave. The autoclave was taken out of the glove box and heated at 220° C. for 48 h.

After the solvothermal treatment, the autoclave was cooled down. The resulting milky suspension was centrifuged, and a white precipitate was collected and re-dispersed in ethanol. The precipitation-dispersion cycle was repeated for three times until the white precipitate was thoroughly washed with ethanol. The BaTiO₃ nanocrystals were suspended in ethanol solvent for storage or further use. The solvothermal process reaches a high yield above 90% based on the recovery from metal barium.

The aggregate-free nature of BaTiO₃ nanocrystals can offer advantages in coating and functionalization of the crystal surface with a variety of surfactants, polymers. For example, a simple solution processing involving the reaction between BaTiO₃ nanocrystals (wet phase) and oleic acid at elevated temperature can provide strong oleic acid binding to the crystal surface, allowing the tailoring of the surface polarity and the crystal solubility in either non-polar solvents (such as hexane, toluene) or polar solvents (such as ethanol, methanol).

To introduce oleic acid as surface ligands, the BaTiO₃ nanocrystals were first collected from the ethanol suspension by centrifugation, and the wet white precipitate was mixed with access amount of oleic acid and stirred at temperature of 80° C. for 1 h to induce strong oleic acid bonding to the crystal surface. The extra amount of oleic acid was washed away with ethanol by repeating a precipitation-dispersion process for three times. The resulting oleic acid-coated nanocrystals were re-dispersed in hexane to obtain a homogeneous and transparent/semitransparent suspension. The stable and homogeneous suspension allows facile film processing and integration with other media.

The X-ray powder diffraction (XRD) of samples (prepared by drop coating nanocrystal suspension on a glass substrate) was measured on an Inel Multipurpose diffractometer using Cu Kα radiation. Transmission Electron Microscopy (TEM) is performed on a JEOL 100CX microscope. The samples were deposited onto a perforated carbon foil supported on a copper grid.

### Example 9

### Thin Film Fabrication Based on BaTiO₃ Nanocrystals

BaTiO₃ nanocrystal thin films were prepared on Si substrates by spin coating of hexane or ethanol suspension of BaTiO₃ nanocrystals at a spin rate of ˜1500 rpm for 1 min. Multiple spin coatings were applied to achieve various thin film thickness. In another case, the BaTiO₃ nanocrystal thin films were prepared by adding several drops of the solution on substrates followed by drying at room temperature. The thin films composed of oleic acid-coated BaTiO₃ were subject to heating treatment at 400° C. to remove the organic coating and solidify the films, if necessary. The thin films prepared from uncoated BaTiO₃ nanocrystals (ethanol suspension) were baked at 60° C. for 12 h to remove the solvent residual and to become stabilized.

### CITED DOCUMENTS

(1) B. Jaffe, W. R. Cook, and H. Jaffe: Piezoelectric Ceramics, (Academic Press, New York, N.Y., USA, 1971), pp. 1-4.
(2) F. Jona, and G. Shirane: Ferroelectric Crystals, (Pergamon Press, New York, N.Y., USA, 1962), pp. 108-203.
(3) N. A. Hill: Why are there so few magnetic ferroelectrics? J. Phys. Chem. B., 104, 6694 (2000).
(4) D. E. Kotecki, J. D. Baniecki, H. Shen, R. B. Laibowitz, K. L. Saenger, J. J. Lian, T. M. Shaw, S. D. Athavale, C. Cabral, P. R. Duncombe, M. Gutsche, G. Kunkel, Y. J. Park, Y. Y. Wang, and R. Wise: (Ba,Sr)TiO3 dielectrics for future stacked-capacitor DRAM. IBM J. Res. Devel., 43, 367 (1999).
(5) B. H. Park, B. S. Kang, S. D. Bu, T. W. Noh, and W. Jo: Lanthanum-substituted bismuth titanate for use in non-volatile memories. Nature, 401, 682 (1999).
(6) J. F. Scott: Ferroelectric Memories, (Springer-Verlag, Berlin, Germany 2000), Vol. 3.
(7) D-J. Kim, J-P. Maria, A. I. Kingon, and S. K. Streiffer: Evaluation of intrinsic and extrinsic contributions to the piezoelectric properties of Pb(Zrl-xTX)O-3 thin films as a function of composition. J App. Phys., 93, 5568 (2003).
(8) L. E. Brus, and J. K. Trautman: Nanocrystals and Nano-Optics. Philos. Trans. R. Soc. London Ser. A-Math. Phys. Eng. Sci., 353, 313 (1995).
(9) A. P. Alivisatos: Semiconductor clusters, nanocrystals, and quantum dots. Science, 271, 933 (1996).
(10) J. R. Heath: Nanoscale materials. Acc. Chem. Res., 32, 388 (1999).
(11) P. Ghosez, and K. M. Rabe: Microscopic model of ferroelectricity in stress-free PbTiO3 ultrathin films. Appl. Phys. Lett., 76, 2767 (2000).
(12) A. Rudiger, T. Schneller, A. Roelofs, S. Tiedke, T. Schmitz, and R. Waser: Nanosize ferroelectric oxide—tracking down the superparaelectric limit, Appl. Phys. a-Mater., 80, 1247 (2005).
(13) N. A. Spaldin: Fundamental size limits in ferroelectricity. Science, 304, 1607 (2004).
(14) J. Zhu, L. Zheng, W. B. Luo, Y. R. Li, and Y. Zhang: Microstructural and electrical properties of BaTiO₃ epitaxial films on SrTiO₃ substructures with a LaNiO₃ conductive layer as a template, J. Phys. D: Appl. Phys., 39, 2438 (2006).
(15) I. B. Misirlioglu, S. P. Alpaya, F. Heb, and B. O. Wells: Stress induced monoclinic phase in epitaxial BaTiO₃ on MgO, J. Appl. Phys., 99, 104103 (2006).
(16) J. Q. He, E. Vasco, R. Dittmann, and R. H. Wang: Growth dynamics and strain relaxation mechanisms in BaTiO3 pulsed laser deposited on SrRuO₃/SrTiO₃, Physical Review B, 73, 125413 (2006).
(17) S. Y. Wang, B. L. Cheng, C. Wang, S. Y. Dai, K. J. Jin, Y. L. Zhou, H. B. Lu, Z. H. Chen, and G. Z. Yang: Raman spectroscopy studies of Ce-doping effects on Ba_{0.5}Sr_{0.5}TiO₃ thin films, J. Appl. Phys., 99, 013504 (2006).
(18) X. H. Zhu, H. L. W. Chan, C.-L. Choy, K.-H. Wong, D. Hesse: A comparative microstructural study of compositionally up- and down-graded (Ba, Sr)TiO₃ thin films epitaxially grown on (La, Sr)CoO₃-covered MgO(100) substrates by pulsed laser
(19) O. Trithaveesak, J. Schubert, and C. Buchal: Ferroelectric properties of epitaxial BaTiO₃ thin films and heterostructures on different substrates, J. Appl. Phys., 98, 114101 (2005).
(20) C.-V. Cojocaru, C. Harnagea, F. Rosei, A. Pignolet, M. A. F. van den Boogaart and J. Brugger: Complex oxide nanostructures by pulsed laser deposition through nanostencils, Appl. Phys. Lett., 86, 183107 (2005).
(21) Y. W. Cho, S. K. Choi, and G. Venkata Rao: The influence of an extrinsic interfacial layer on the polarization of sputtered BaTiO₃ film, Appl. Phys. Lett., 86, 202905 (2005).
(22) H.-N. Tsai, Y.-C. Liang, and H.-Y. Lee: Characteristics of sputter-deposited BaTiO₃/SrTiO₃ artificial superlattice films on an LaNiO₃-coated SrTiO₃ substrate, J. Crystal Growth, 284, 65 (2005).
(23) Y. K. Vayunandana Reddy, D. Mergel, S. Reuter, V. Buck, and M. Sulkowski: Structural and optical properties of BaTiO₃ thin films prepared by radio-frequency magnetron sputtering at various substrate temperatures, J. Phys. D: Appl. Phys., 39, 1161 (2006).
(24) J. H. Ma, Z. M. Huang, X. J. Meng, S. J. Liu, X. D. Zhang, J. L. Sun, J. Q. Xue, J. H. Chu and J. Li: Optical properties of SrTiO₃ thin films deposited by radio-frequency magnetron sputtering at various substrate temperatures, J. Appl. Phys., 99, 033515 (2006).
(25) F. El Kamel, P. Gonon, and F. Jomni: Electrical properties of low temperature deposited amorphous barium titanate thin films as dielectrics for integrated capacitors, Thin Solid Films, 504, 201 (2006).
(26) Y. A. Boikov, and T. Claeson: Microstructure and dielectric parameters of epitaxial SrRuO₃/BaTiO₃/SrRuO₃ heterostructures, J. Appl. Phys., 89, 5053 (2001).
(27) A. Petraru, J. Schubert, M. Schmid, and C. Buchal: Ferroelectric BaTiO₃ thin-film optical waveguide modulators, Appl. Phys. Lett., 81, 1375 (2002).
(28) M. H. Frey, and D. A. Payne: Synthesis and processing of barium-titanate ceramics from alkoxide solutions and monolithic gels. Chem. Mater., 7, 123 (1995).
(29) R. N. Viswanath, and S. Ramasamy: Preparation and ferroelectric phase transition studies of nanocrystalline BaTiO₃. Nanostruct. Mater., 8, 155 (1997).
(30) K. M. S. Khalil: Low temperature evolution of crystalline BaTiO₃ from alkali-metal free precursor using sol-gel process. Mater. Res. Innovations, 2, 256 (1999).
(31) (Special Issue) Sol-Gel Processing of Ferroelectric Materials. J. Sol.-Gel. Sci. Technol., (Springer Netherlands, 1999), 16, Numbers 1-2, pp. 1±193.
(32) H. Matsuda, N. Kobayashi, T. Kobayashi, K. Miyazawa, and M. Kuwabara: Room-temperature synthesis of crystalline barium titanate thin films by high-concentration sol-gel method. J. Non-Cryst. Solids, 271, 162 (2000).
(33) K. M. Hung, W. D. Yang, and C. C. Huang: Preparation of nanometer-sized barium titanate powders by a sol-precipitation process with surfactants. J Eur. Ceram. Soc., 23, 1901 (2003).
(34) G. Fan, L. Huangpu, and X. He: Synthesis of single-crystal BaTiO₃ nanoparticles via a one-step sol-precipitation route. J. Crystal Growth, 279, 489 (2005).
(35) B. A. Hernandez, K. S. Chang, E. R. Fisher, and P. K. Dorhout: Sol-gel template synthesis and characterization of BaTiO₃ and PbTiO₃ nanotubes. Chem. Mater., 14, 480 (2002).
(36) Y—S. Her, E. Matijevic, and M. C. Chon: Preparation of well-defined colloidal barium-titanate crystals by the controlled double-jet precipitation. J. Mater. Res., 10, 3106 (1995).
(37) M. Z-C. Hu, G. A. Miller, E. A. Payzant, and C. J. Rawn: Homogeneous (co)precipitation of inorganic salts for synthesis of monodispersed barium titanate particles. J Mater. Sci., 35, 2927 (2000).
(38) S. Wada, T. Tsurumi, H. Chikamori, T. Noma, and T.Suzuki: Preparation of nm-sized BaTiO₃ crystallites by a LIDS method using a highly concentrated aqueous solution. J. Cryst. Growth, 229, 433 (2001).
(39) H. R. Xu and L. Gao: Tetragonal nanocrystalline barium titanate powder: Preparation, characterization, and dielectric properties. J. Am. Ceram. Soc., 86, 203 (2003).
(40) P. Duran, F. Capel, D. Gutierrez, J. Tartaj, M. A. Banares, and C. Moure: Metal citrate polymerized complex thermal decomposition leading to the synthesis of BaTiO₃: effects of the precursor structure on the BaTiO₃ formation mechanism. J. Mater. Chem., 11, 1828 (2001).
(41) H. Rumpf, H. Modrow, J. Hormes, H. J. Glasel, E. Hartmann, E. Erdem, R. Bottcher, and K. H. Hallmeier: Preparation of nanocrystalline BaTiO₃ characterized by in situ X-ray absorption spectroscopy. J. Phys. Chem. B, 105, 3415 (2001).
(42) P. R. Arya, P. Jha, and D. Ganguli: Synthesis, characterization and dielectric properties of nanometersized barium strontium titanates prepared by the polymeric citrate precursor method. J. Mater. Chem., 13, 415 (2003).
(43) S. O′Brien, L. Brus, and C. B. Murray: Synthesis of monodisperse nanoparticles of barium titanate: Toward a generalized strategy of oxide nanoparticle synthesis. J. Am. Chem. Soc., 123, 12085 (2001).
(44) J. J. Urban, W. S. Yun, Q. Gu, and H. Park: Synthesis of single-crystalline perovskite nanorods composed of barium titanate and strontium titanate. J. Am. Chem. Soc., 124, 1186 (2002).
(45) I. J. Clark, T. Takeuchi, N. Ohtori, and D. C. Sinclair: Hydrothermal synthesis and characterisation of BaTiO₃ fine powders: precursors, polymorphism and properties. J. Mater. Chem., 9, 83 (1999).
(46) P. K. Dutta and J. R. Gregg: Hydrothermal synthesis of tetragonal barium-titanate. Chem. Mater., 4, 843 (1992).
(47) R. I. Walton, F. Millange, R. I. Smith, T. C. Hansen, and D. O′Hare: Real time observation of the hydrothermal crystallization of barium titanate using in situ neutron powder diffraction. J. Am. Chem. Soc., 123, 12547 (2001).
(48) E. Ciftci, M. N. Rahaman, and M. Shumsky: Hydrothermal precipitation and characterization of nanocrystalline BaTiO₃ particles. J. Mater. Sci., 36, 4875 (2001).
(49) M-S. Zhang, J. Yu, J. Chu, D. Chen, and W. Chen: Microstructures and photoluminescence of barium titanate nanocrystals synthesized by the hydrothermal process. J. Mater. Process. Technol., 137, 78 (2003).
(50) X. Wang, J. Zhuang, Q. Peng, and Y. Li: A general strategy for nanocrystal synthesis. Nature, 437, 121 (2005).
(51) T. Yan, Z-G. Shen, J-F. Chen, X-L. Liu, X. Tao, and J. Yun: Synthesis of well-isolated barium titanium trioxide nanocubes. Chemistry letters, 34, 1196 (2005).
(52) M. Niederberger, N. Pinna, J. Polleux, and M. Antonietti: A general soft-chemistry route to perovskites and related materials: Synthesis of BaTiO₃, BaZrO₃, and LiNbO₃ nanoparticles. Angew Chem Int Edit., 43, 2270 (2004).
(53) M. Niederberger, G. Garnweitner, N. Pinna, and M. Antonietti: Nonaqueous and halide-free route to Crystalline BaTiO₃, SrTiO₃, and (Ba,Sr)TiO₃ nanoparticles via a mechanism involving C—C bond formation. J. Am. Chem. Soc., 126, 9120 (2004).
(54) N. Nuraje, K. Su, M. Haboosheh, J. Samson, E. P. Manning, N-L. Yang, and H. Matsui: Room temperature synthesis of ferroelectric barium titanate nanoparticles using peptide nanorings as templates. Advanced Materials, 18, 807 (2006).
(55) X. Wu, L. Zou, S. Yang, and D. Wang: Structural characterizations of organo-capped barium titanate nanoparticles prepared by the wet chemical route. J Colloid Interface Sci., 239, 369 (2001).
(56) E. V. Shevchenko, D. V. Talapin, N. A. Kotov, S. O′Brien, and C. B. Murray: Structural diversity in binary nanoparticle superlattices. Nature, 439, 55 (2006).
(57) F. X. Redl, K. S. Cho, C. B. Murray, and S. O′Brien: Three-dimensional binary superlattices of magnetic nanocrystals and semiconductor quantum dots. Nature, 423, 968 (2003).
(58) R. C. Mehrotra, A. Singh, and S. Sogani: Homometallic and heterometallic alkoxides of group 1,2, and 12 metals. Chem. Soc. Rev., 23, 215 (1994).
(59) Z. Chen, L. Huang, J. He, Y. Zhu, S. O′Brien, A new nonhydrolytic route to synthesize crystalline BaTiO3 nanocrystals with surface capping ligands, J. Mater. Res. 2006, 21, 3187.

All publications, patents, and patent documents cited in the specification are incorporated by reference herein, as though individually incorporated by reference. In the case of any inconsistencies, the present disclosure, including any definitions therein, will prevail. The invention has been described with reference to various non-limiting examples and embodiments. However, it should be understood that many variations and modifications can be made while remaining within the spirit and scope of the present invention.

What is claimed is:

1. A nanocrystalline form of a metal oxide, the form comprising a plurality of nanocrystals, the plurality of nanocrystals having a narrow size distribution and an average particle diameter ranging from about 1 nm to about 100 nm, the nanocrystals comprising a metal oxide of formula M¹ₓO_{z}, a mixed metal oxide of the perovskite type of formula M²M³O₃, or a complex mixed metal oxide of the formula M⁴ₓM⁵_{y}O_{z}, wherein all of M¹-M⁵ are independently selected ions of metallic elements.

2. The nanocrystalline form of claim 1 wherein the narrow size distribution is a substantially monodisperse size distribution.

3. The nanocrystalline form of claim 1 further comprising ions of additional metallic elements other than M¹-M⁵ in a crystal lattice of the metal oxide, mixed metal oxide, or complex mixed metal oxide.

4. The nanocrystalline form of claim 3 wherein the ions of additional metallic elements comprise ions of zirconium, yttrium, or rare earth metals.

5. The nanocrystalline form of claim 1 wherein an organic coating material is disposed on a surface of the nanocrystals to provide capped or coated nanocrystals.

6. The nanocrystalline form of claim 1 wherein the nanocrystals comprise a metal oxide of formula M¹ₓO_{z}.

7. The nanocrystalline form of claim 6 wherein M¹ is titanium, zirconium, hafnium, vanadium, niobium, tantalum, tungsten, manganese, iron, cobalt, nickel, copper, zinc, gallium, indium, tin or cerium.

8. The nanocrystalline form of claim 6 wherein x is 1 to about 3 and z is 1 to about 6.

9. The nanocrystalline form of claim 6 comprising zinc oxide, titanium oxide, or zirconium oxide.

10. The nanocrystalline form of claim 1 wherein the nanocrystals comprise a mixed metal oxide of the perovskite type of formula M²M³O₃.

11. The nanocrystalline form of claim 10 wherein M² comprises barium, strontium, calcium, lithium, lead, yttrium, bismuth, lanthanum, or a rare earth metal, or wherein M³ comprises titanium, zirconium, iron, copper, manganese, cerium, or cobalt; or both.

12. The nanocrystalline form of claim 10 comprising barium titanate, strontium titanate, calcium titanate, barium strontium titanate, barium lanthanum, lithium lanthanum titanate, lead titanate, lead zirconium titanate, barium zirconate, lead zirconate, yttrium ferrite, bismuth ferrite, yttrium barium copper oxide, lanthanum manganese oxide, strontium cerium oxide, or a rare earth cobalt oxide or any combination thereof.

13. The nanocrystalline form of claim 1 wherein the nanocrystals comprise a complex metal oxide of the formula M⁴ₓM⁵_{y}O_{z}.

14. The nanocrystalline form of claim 13 wherein M⁴ comprises indium, lithium, bismuth or yttrium, or wherein M⁵ comprises tin, niobium, or iron; or both.

15. The nanocrystalline form of claim 13 wherein x is 1 to about 3, y is 1 to about 5, or z is 3 to about 12, or any combination thereof.

16. The nanocrystalline form of claim 13 comprising indium tin oxide, lithium niobium oxide, or a garnet, or any combination thereof.

17. The nanocrystalline form of claim 5 wherein the organic coating material comprises an alkanoic acid, a saturated or unsaturated fatty acid, decanoic acid, oleic acid, an alkylamine, a fatty amine, oleylamine, an alkanol, a fatty alcohol, or oleyl alcohol, or a combination thereof.

18. A method of preparation of the metal oxide nanocrystalline form of claim 1, comprising contacting an metalorganic precursor, wherein the metalorganic precursor comprises a single metallic element or more than one metallic element, and a liquid substance comprising an alcohol at an elevated temperature of less than about 350° C., to provide the plurality of metal oxide nanocrystals having a narrow size distribution.

19. The method of claim 18 wherein the metalorganic precursor comprises a metal alkoxide, a metal carboxylate, or a metal complex such as a metal acetoacetonate.

20. The method of claim 18 further comprising, after contacting the metalorganic precursor and the liquid substance, then, contacting with a reagent, then, collecting the plurality of metal oxide nanocrystals, wherein collecting comprises centrifuging.

21. The method of claim 18 wherein the metalorganic precursor comprises a metallic element selected from the group consisting of titanium, zirconium, hafnium, vanadium, niobium, tantalum, tungsten, manganese, iron, cobalt, nickel, copper, zinc, gallium, indium, tin and cerium, and, optionally, further comprises a second metallic element selected from the group consisting of barium, strontium, calcium, lithium, lead, yttrium, bismuth, lanthanum, a rare earth metal, titanium, zirconium, iron, copper, manganese, cerium, and cobalt.

22. The method of claim 21 wherein the metalorganic precursor comprises titanium and barium.

23. The method of claim 18 wherein the metalorganic precursor comprises a titanium alkoxide, titanium acetate, or titanium acetoacetonate and a barium alkoxide, barium acetate, or barium acetoacetonate.

24. The method of claim 18 wherein the liquid substance comprises water, or aqueous alkali, aqueous sodium hydroxide, aqueous potassium hydroxide, or tetrapropylammonium hydroxide.

25. The method of claim 18 comprising forming the metal oxide precursor solution by contacting a first metal alkoxide and a second metal alkoxide and a liquid substance comprising an alcohol at an elevated temperature of less than about 350° C. to form the nanocrystalline form.

26. The method of claim 25 further comprising, after contacting the first metal alkoxide and second metal alkoxide and the liquid substance comprising an alcohol at an elevated temperature of less than about 350° C., then, contacting with a reagent.

27. The method of claim 26 wherein an amount of the reagent added to the metal oxide precursor solution comprises up to about 20% of a volume of the precursor solution.

28. The method of claim 26 wherein the reagent comprises ethanol, isopropanol, or water, or any combination thereof.

29. The method of claim 26 wherein the reagent comprises an alkali, sodium hydroxide, or potassium hydroxide, or tetrapropylammonium hydroxide.

30. The method of any claim 18 wherein the elevated temperature is about 80° C. to about 230° C.

31. The method of claim 18 wherein the metalorganic precursor and liquid substance comprising an alcohol are contacted under a pressure of about 20 atm to about 30 atm.

32. The method of claim 18 further comprising contacting the metalorganic precursor and the liquid substance with an organic coating material.

33. The method of claim 32 wherein the organic coating material comprises an alkanoic acid, a saturated or unsaturated fatty acid, decanoic acid, oleic acid, an alkylamine, a fatty amine, oleylamine, an alkanol, a fatty alcohol, or oleyl alcohol, or a combination thereof.

34. The method of claim 18 comprising a method of preparation of a plurality of nanocrystals comprising BaTiO₃, the plurality having a narrow size distribution and an average nanocrystal diameter of about 2-80 nm, the method comprising:
contacting a barium metalorganic precursor and a titanium alkoxide to provide a bimetallic precursor solution; then,
contacting the bimetallic precursor solution and a liquid substance comprising an alcohol at an elevated temperature of less than about 350° C. for a period of time to provide the plurality of nanocrystals comprising BaTiO₃.

35. The method of claim 34 comprising dissolving barium metal in an alcohol to provide the barium metalorganic precursor, wherein alcohol is benzyl alcohol, ethanol, or isopropanol.

36. The method of claim 34 wherein the barium metalorganic precursor is barium ethoxide, barium isopropoxide, or barium benzyloxide.

37. The method of claim 34 wherein the titanium alkoxide comprises titanium isopropoxide.

38. The method of claim 34 wherein the liquid substance further comprising an organic coating material comprising a hydrophobic long chain amine, oleylamine, a fatty acid, oleic acid, decanoic acid, a fatty alcohol, oleyl alcohol, or any combination thereof.

39. The method of claim 118 wherein the nanocrystals comprise decanoic acid capped BaTiO₃ nanocrystals of about 6-10 nm size, or wherein the nanocrystals comprise oleic acid capped BaTiO₃ nanocrystals of about 3-5 nm size, or wherein the nanocrystals comprise oleic acid capped BaTiO₃ nanoparticle/nanorod mixture of about 10-20 nm size, or wherein the nanocrystals comprise oleic acid capped BaTiO₃ nanocrystals of about 2-3 nm size.

40. A substantially homogeneous dispersion of the nanocrystalline form of claim 1 in a liquid.

41. A substantially homogeneous dispersion of a nanocrystalline form prepared by the method of claim 18 in a liquid.

42. The dispersion of claim 40 wherein the nanocrystals are capped or coated and the non-polar organic solvent comprises hexane or toluene, or a mixture thereof.

43. The dispersion of claim 40 further comprising a surfactant, a polymer, a liquid crystal forming material, a phospholipid, or a mixture thereof; and optionally further comprising a matrix precursor.

44. The dispersion of claim 43 wherein the matrix precursor comprises an organic matrix precursor adapted for polymerization for formation of an organic matrix, or an inorganic matrix precursor adapted for formation of an inorganic matrix, or a mixed organic/inorganic matrix precursor adapted for formation of an organic/inorganic matrix, or any mixture thereof.

45. The dispersion of claim 44 wherein the organic matrix comprises a polyacrylate, a poly(methyl methacrylate), a polyurethane, or a block organic copolymer; or wherein the inorganic matrix comprises silica; or wherein the organic/inorganic matrix comprises interpenetrating networks of silica and an organic polymer; or any combination thereof.

46. A method of forming a film comprising a plurality of metal oxide nanocrystals, the method comprising disposing a dispersion of the nanocrystalline form of claim 1 on a substrate, then, removing the liquid substance, to provide the film disposed on the substrate.

47. The method of claim 46 wherein the film further comprises a matrix.

48. The method of claim 46 wherein the substrate comprises an electrically insulating, conductive or semi-conductive material.

49. The method of claim 48 wherein the substrate is a surface composed of a solid material comprising silicon, silicon nitride, silica, diamond, or an organic plastic.

50. The method of any 46 wherein the film is substantially free of voids or the film is adapted to comprise a particular proportion of voids.

51. A film comprising the nanocrystalline form of claim 1.

52. The film of claim 51 having a thickness of about 10 nm to about 1 millimeter.

53. The film of claim 51 having a dielectric constant of greater than about ten.

54. The film of claim 51 further comprising an organic component used in generation of a mesoporous material.

55. The film of claim 54 wherein the organic compound used in generation of a mesoporous material comprises MCM-41, MCM-48, SBA-15, or SBA-16.

56. The film of claim 51 wherein the film is substantially free of voids or the film is adapted to comprise a particular proportion of voids.

57. The film of claim 51 wherein the film, comprising a type of the nanocrystalline form of claim 1, substantially retains electrical properties, density properties, spectral properties, hardness properties or scratch resistance, or thermal properties, or any combination thereof, of the respective type of nanocrystalline form.

58. A dielectric layer comprising the film of claim 51.

59. A capacitor or an ultracapacitor comprising the dielectric layer of claim 58.

60. The capacitor or ultracapacitor of claim 59 wherein the dielectric layer is disposed on or within a mesoporous structure.

61. A field effect transistor comprising the dielectric layer of claim 58.

62. The field effect transistor of claim 61, comprising:
a semiconductor device, comprising:
a thin film gate dielectric comprising barium titanate nanoparticles of approximately uniform size that is smaller than a domain size associated with ferroelectric hysteresis, wherein a dielectric constant of the dielectric is greater than about 10; and
an organic semiconductor region.

63. The transistor of claim 62, comprising a buffer layer between the dielectric and the organic semiconductor.

64. The transistor of claim 63, wherein the buffer layer comprises parylene C.

65. The transistor of claim 64, wherein the organic semiconductor comprises pentacene.

66. The apparatus of claim 62, wherein the dielectric constant is at least about 40.

67. A display device comprising the dielectric layer of claim 58.

68. A memory device comprising the dielectric layer of claim 58 such that the dielectric layer is disposed as a gate dielectric in a transistor in a memory cell in an array of memory cells of the memory device.

69. A memory device comprising the dielectric layer of claim 58 such that the dielectric layer is disposed as a capacitor dielectric in a capacitor coupled to a transistor in a memory cell in an array of memory cells of the memory device, the capacitor structured in the memory cell as a data storage unit.

70. The memory device of claim 69, wherein the nanocrystals are ferroelectric.

71. The memory device of claim 69, wherein an electrode of the capacitor is disposed as a gate of the transistor.

72. An opto-electronic device comprising the dielectric layer of claim 58.

73. The opto-electronic device of claim 72, wherein the dielectric layer is arranged in a switching element thereof.

74. A coating layer comprising the nanocrystalline form of claim 1.

75. The coating layer of claim 74 comprising zinc oxide, titanium oxide, or zirconium oxide.

76. The coating layer of claim 75 further comprising a cream or oil adapted for application to human skin.
